(19) United States
(12) Patent Application Publication
(10) Pub. No.: US 2024/0383941 A1
(43) Pub. Date: Nov. 21, 2024

(54) 3-ALPHA SUBSTITUTED 3-BETA-HYDROXY-17-OXIMATED ANDROSTANE COMPOUND FOR MODULATION OF THE ALPHA-3 SUBTYPE OF THE GABAA RECEPTOR

(71) Applicant: UMECRINE AB, Umeå (SE)

(72) Inventors: Torbjörn BÄCKSTRÖM, Umeå (SE); Gianna RAGAGNIN, Umeå (SE); Jessica SJÖSTEDT, Gideå (SE)

(73) Assignee: UMECRINE AB, Umeå (SE)

(21) Appl. No.: 18/707,708

(22) PCT Filed: Nov. 10, 2022
(86) PCT No.: PCT/EP2022/081517

§ 371 Date: May 6, 2024

(30) Foreign Application Priority Data
Nov. 10, 2021 (EP) 21207633.5

(51) Int. Cl. C07J 41/00; A61K 31/568; A61P 3/10

(52) U.S. Cl. CPC C07J 41/0016; A61K 31/568; A61P 3/10

(57) ABSTRACT

The present disclosure concerns the novel compound 3.alpha.-ethynyl-3.beta.-hydroxy-5.alpha.-androstan-17-methoxime, the medical use thereof and in particular use in the treatment of diseases and disorders associated with an a3 subtype of the GABAA receptor, for example treatment of obesity, hyperphagia disorder, Prader-Willi's syndrome, polycystic ovarian syndrome, and/or diabetes. Said disclosure is also concerned with reducing and/or preventing overweight. Additionally, related pharmaceutical and cosmetic compositions are disclosed.

### TECHNICAL FIELD

The present disclosure concerns novel steroid compounds, the medical use thereof and in particular use in the treatment of diseases and disorders associated with an a3 subtype of the GABA_{A} receptor, for example treatment of obesity, hyperphagia disorder, Prader-Willi's syndrome, polycystic ovarian syndrome, and/or diabetes. Said disclosure is also concerned with reducing and/or preventing overweight. Additionally, related pharmaceutical and cosmetic compositions are disclosed.

### BACKGROUND

Gamma-aminobutyric acid (GABA) is the main inhibitory neurotransmitter in the central nervous system, and in the rest of the body acting on the GABA_{A} and GABA_{B} receptors.

The GABA_{A} receptors are of several subtypes, located in different areas of the brain and are related to different CNS disorders and symptoms. Some GABA_{A} receptors are localized within a synapse (intra-synaptic) while others are located outside a synapse (extra-synaptic). Some GABA_{A} receptor modulating steroids can in physiological concentrations open the extra-synaptic GABA_{A} receptor by themselves (tonic inhibition) but not the intra-synaptic receptors (phasic inhibition). These two types of effects are dependent on different mechanisms on the GABA_{A} receptor and the effects depend in addition on the subunit composition of the receptor. In addition, positive GABA_{A} receptor modulating steroids (GAMS) can enhance the effect of GABA in both extra and intrasynaptic receptors. The receptor subtype α4,β,δ is an extra-synaptic subtype with both tonic and phasic effects when subjected to 3α-hydroxy steroids, such as 3α-hydroxy-5α/β-pregnan-20-one/ol or 3α-hydroxy-5α/β-androstan-17-one/ol.

The GABA system plays an important role in many bodily functions, including the regulation of eating behavior. Many steroid-related CNS disorders or diseases and diabetes have been coupled to GABA signaling. The World Health Organization (WHO) have estimated that today nearly 2 billion adults worldwide, aged 18 years and older, are overweight. Obesity and overweight pose a major risk for chronic diseases, including type 2 diabetes, cardiovascular disease, hypertension and stroke, and certain forms of cancer. While energy balance is key to maintaining a healthy weight, genes are important in determining a person's susceptibility to weight gain.

Positive GABA_{A} receptor modulating steroids (GAMS) are metabolites of the sex and stress hormones pregnanolone, progesterone, deoxycorticosterone, cortisone and cortisol, known as pregnanolones; as well as the metabolites of testosterone, androstanedione and dehydroepiandrosterone, known as androstanes. GAMS have been the subject of various studies, at least partially elucidating their role in the neurological signal system in mammals.

These steroid metabolites induce CNS symptoms and disorders. They may share a 3α-hydroxy group, a 5α or 5β pregnane or androstane steroid body, or a double bond between carbon atoms 4 and 5 and a ketone or hydroxy group on position 17, 20 or 21. Examples of such steroids are 3α-hydroxy-5α/β-pregnan/δ4-pregnen-20-one/ol steroids or 3α-hydroxy-5α/β-androstan/δ4-androsten-17-one/ol steroids, such as allopregnanolone, tetrahydrodeoxycorticosterone and androstanediol. Another example of a GAMS is tetrahydrodeoxycorticosterone (THDOC).

It was been shown that an abrupt reduction of food intake is seen after hampering GABAergic transmission. In animal studies, local application of GABA_{A}-receptor agonists in key areas of feeding regulation in the brain has been shown to induce hyperphagia disorder. Excessive food intake is a well-known effect of GABA_{A} receptor modulating steroids (GAMS). This has been shown in both animal and human studies.

The α3 subtypes are known to regulate feeding, hunger, and satiety. In the brain, mainly the α3β3γ2 receptor subtype is expressed.

As the 3α-hydroxy-pregnane/androstane steroids are endogenously produced and are metabolites of steroid hormones essential for life, their production cannot easily be interrupted. It was established previously that 3α-hydroxy-5α/β steroids may cause CNS disorders through the three possible mechanisms of a) direct action, b) tolerance induction, and/or c) withdrawal effect. These steroids are produced in high amounts during several days to years in specific disorders for example in obesity, hyperphagia disorder, Prader-Willi's syndrome, polycystic ovarian syndrome, diabetes, during acute and chronic stress, the luteal phase of the menstrual cycle and during pregnancy. They are also continuously produced within the brain in high amounts at certain disorders. Their production is locally regulated. In certain disorder the specific receptor subtype is downregulated or not expressed at all. In such situations, the body compensates by expressing another receptor subtype. The α4,β,δ receptor type is often then overexpressed. The α4,β,δ receptor subtype is very sensitive to GAMS.

U.S. Pat. Nos. 5,232,917, 5,925,630, 5,939,545, 6,143,736 and 6,277,838 disclose a number of 3α-hydroxy steroids and 3β steroids. WO 99/45931 and WO 03/059357 disclose antagonistic effects of steroids. WO 08/063128 discloses a number of steroids, such as 3α-ethynyl-3β-hydroxy-5α-androstan-17-oxime.

Wang et al. 2000 (Acta Physiol Scand 169, 333-341) and Wang et al. 2002 (J Neurosci 22(9):3366-75) discloses antagonistic effects of 3β-hydroxy-5α-pregnan-20-one and other 3β-hydroxy 5α/β pregnane steroids.

WO 09/142594 discloses 3α-ethynyl-3β-hydroxy-5α-androstan-17-one and teaches that this steroid has no effect as a GAMSA or GAMS (see table 3 in WO 09/142594).

Many diseases and disorders are associated with GABA_{A} receptor signalling and there is a large need in the field to identify specific modulators of GABA signal transduction, which modulate signalling via desired subtypes of GABA_{A} receptors. In particular, as obesity and obesity related disorders are a large heath problem in the worlds, there is a great need to provide ways of treatment, alleviation and/or prevention of the obesity and obesity related disorders. It remains a challenge to suppress the effects on GAMS and obtain blockers thereof useful in therapy for example, in order to treat reduce the excessive food intake. Specific blockers, in particular blocker of α3 subunit of GABA_{A} receptors, are therefore needed. In addition, it remains a challenge to find compounds that are physiologically safe and suitable for pharmaceutical use, and which additionally are applicable in physiologically acceptable doses with reasonable time intervals, for the treatment of said disorders.

### DISCLOSURE OF THE INVENTION

It is an object of the present invention to provide compounds that reduce or at least partly overcome challenges in the prior art. It is an object of the present invention to provide novel compounds which have the ability to suppress GABA signalling and/or positive GABA_{A} receptor modulating steroids (GAMS). In particular, the object of the present invention is to provide novel compounds which antagonizing GABA signaling via the α3 GABA_{A} receptor subtype at least. An object is to provide such compounds which may be useful as medicaments and/or in non-therapeutic (in other words cosmetic) applications.

It is an object of the present invention to provide means for treatment, alleviation and/or prevention of a steroid-related CNS disorder or disease, an autoimmune disease and/or diabetes. Such steroid-related CNS disorder may for example be obesity, hyperphagia disorders and diseases or disorders associated with obesity or related to obesity. It is an object to provide means for reducing weight in a mammal by suppressing positive GABA_{A} receptor modulating steroids (GAMS). In particular, it is an object to provide compounds useful in therapies to treat said disorders. It is also an object of the present invention to provide non-therapeutic treatments of overweight and overeating.

The present inventors have identified the novel the steroids compound 3α-ethynyl-3β-hydroxy-5α-androstan-17-methoxime as shown in Formula 1

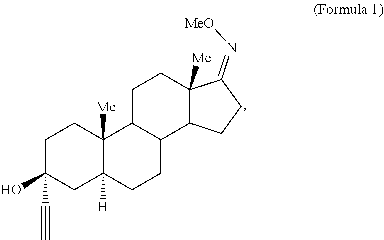 [SMILES: C#C[C@]1(O)CC[C@]2(C)C3CC[C@]4(C)/C(=N\OC)CCC4C3CC[C@H]2C1]

3α-ethyl-3β-hydroxy-5α-androstan-17-oxime as shown in Formula 2

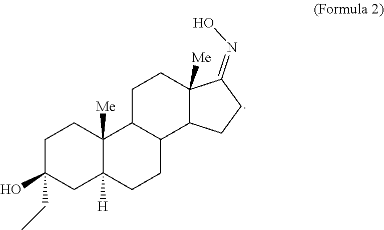 [SMILES: CC[C@]1(O)CC[C@]2(C)C3CC[C@]4(C)/C(=N\O)CCC4C3CC[C@H]2C1]

The inventors show that 3α-ethynyl-3β-hydroxy-5α-androstan-17-methoxime and 3α-ethyl-3β-hydroxy-5α-androstan-17-oxime provide an antagonistic effect on GAMS enhancement of the α3 subtypes of the GABA_{A}-receptor-chloride ionophore complex. Therefore, these compounds blocks the negative effects of GAMS. The compounds thereby acts as a GAMS antagonist (GAMSA). As such, the present inventors show that 3α-ethynyl-3β-hydroxy-5α-androstan-17-methoxime and 3α-ethyl-3β-hydroxy-5α-androstan-17-oxime can be used in the treatment of GAMS-related and/or steroid-induced disorders or diseases of the central nervous system (CNS) as well as diabetes. These and other objects are achieved in full, or at least in part, by aspects of the inventive concepts as disclosed herein.

In a first aspect, there is provided compound selected from the group consisting of
3α-ethynyl-3β-hydroxy-5α-androstan-17-methoxime as shown in Formula 1, and
3α-ethyl-3β-hydroxy-5α-androstan-17-oxime as shown in Formula 2, or
a pharmaceutically acceptable salt, hydrate, prodrug or solvate thereof or a cosmetically acceptable salt, hydrate, precursor or solvate thereof, such as a pharmaceutically acceptable salt, hydrate or solvate thereof or a cosmetically acceptable salt, hydrate or solvate thereof. Thus, the encompassed by the present disclosure are pharmaceutically acceptable salts, hydrates, prodrugs or solvates as well as cosmetically acceptable salts, hydrates, precursors or solvates of 3α-ethynyl-3β-hydroxy-5α-androstan-17-methoxime and pharmaceutically acceptable salts, hydrates, prodrugs or solvates as well as cosmetically acceptable salts, hydrates, precursors or solvates of 3α-ethyl-3β-hydroxy-5α-androstan-17-oxime.

In one embodiment, said compound is 3α-ethynyl-3β-hydroxy-5α-androstan-17-methoxime or a pharmaceutically acceptable salt, hydrate, prodrug or solvate thereof, such as pharmaceutically acceptable salt, hydrate or solvate thereof. In another embodiment said compound is 3α-ethynyl-3β-hydroxy-5α-androstan-17-methoxime or a cosmetically acceptable salt, hydrate, precursor or solvate thereof, such as cosmetically acceptable salt, hydrate or solvate thereof.

In one embodiment, said compound is 3α-ethyl-3β-hydroxy-5α-androstan-17-oxime, or a pharmaceutically acceptable salt, hydrate, prodrug or solvate thereof, such as pharmaceutically acceptable salt, hydrate or solvate thereof. In another embodiment said compound is 3α-ethyl-3β-hydroxy-5α-androstan-17-oxime, or a cosmetically acceptable salt, hydrate, precursor or solvate thereof, such as cosmetically acceptable salt, hydrate or solvate thereof.

The compounds disclosed herein exist as optical isomers and with deuterium or tritium instead of hydrogen; the invention encompasses compounds with all isotopes. In the synthesis of said compounds, individual isomers may need to be separated by chromatographic techniques and/or by other separations methods. Thus, in one particular embodiment, wherein said compound or a pharmaceutically acceptable salt, hydrate, prodrug or solvate thereof or a cosmetically acceptable salt, hydrate, precursor or solvate thereof comprises ³H isotopes of hydrogen. In another embodiment, said compound or a pharmaceutically acceptable salt, hydrate, prodrug or solvate thereof or a cosmetically acceptable salt, hydrate, precursor or solvate thereof comprises ²H isotopes of hydrogen.

3α-ethynyl-3β-hydroxy-5α-androstan-17-methoxime and 3α-ethyl-3β-hydroxy-5α-androstan-17-oxime may form salts which are within the scope of the present invention. Salts which are suitable for use in medicine are those wherein a counterion is pharmaceutically acceptable. Salts which are suitable for use in non-therapeutic uses are those wherein a counterion is cosmetically acceptable.

The skilled person is aware of suitable salts for use in medicine and cosmetic applications. For example, suitable salts according to the invention include those formed with organic or inorganic acids or bases. In particular, suitable salts formed with acids according to the invention include those formed with mineral acids, strong organic carboxylic acids, such as alkanecarboxylic acids of 1 to 4 carbon atoms which are unsubstituted or substituted, for example, by halogen, such as saturated or unsaturated dicarboxylic acids, such as hydroxycarboxylic acids, such as amino acids, or with organic sulfonic acids, such as (C₁-C₄)alkyl or aryl sulfonic acids which are unsubstituted or substituted, for example by halogen. Pharmaceutically and/or cosmetically acceptable acid addition salts include those formed from hydrochloric, hydrobromic, sulphuric, nitric, citric, tartaric, acetic, phosphoric, lactic, pyruvic, acetic, trifluoroacetic, succinic, perchloric, fumaric, maleic, glycolic, lactic, salicylic, oxaloacetic, methanesulfonic, ethanesulfonic, p-toluenesulfonic, formic, benzoic, malonic, naphthalene-2-sulfonic, benzenesulfonic, isethionic, ascorbic, malic, phthalic, aspartic, and glutamic acids, lysine and arginine.

Pharmaceutically and/or cosmetically acceptable base salts include ammonium salts, alkali metal salts, for example those of potassium and sodium, alkaline earth metal salts, for example those of calcium and magnesium, and salts with organic bases, for example dicyclohexylamine, N-methyl-D-glucamine, morpholine, thiomorpholine, piperidine, pyrrolidine, a mono, di- or tri lower alkylamine, for example ethyl, tertbutyl, diethyl, diisopropyl, triethyl, tributyl or dimethylpropylamine, or a mono-,di- or trihydroxy lower alkylamine, for example mono-, di- or triethanolamine. Corresponding internal salts may furthermore be formed.

In one embodiment, said pharmaceutically or cosmetically acceptable salt is a sodium salt. As apparent to a person of skill in the art, other salts may be equally suitable for the present compound. Non-limiting examples of other suitable salts are hydrochloride, sulfate, acetate, phosphate or diphosphate, chloride, potassium, maleate, calcium, citrate, mesylate, nitrate, tartrate and aluminum gluconate.

It should be understood that the compounds according to the present disclosure may be useful as a therapeutic agents in its own right. A direct therapeutic effect may for example be accomplished by provides an antagonistic effect on GAMS enhancement of α3 subtype(s) of the GABA_{A}-receptor-chloride ionophore complex.

Thus in a second aspect of the present there is provided a compound as disclosed herein for use as a medicament. Thus 3α-ethynyl-3β-hydroxy-5α-androstan-17-methoxime, 3α-ethyl-3β-hydroxy-5α-androstan-17-oxime or a pharmaceutically acceptable salt, hydrate, prodrug or solvate of either of said compounds may be used as a medicament.

As explained above, the present inventors show that 33α-ethynyl-3β-hydroxy-5α-androstan-17-methoxime and 3α-ethyl-3β-hydroxy-5α-androstan-17-oxime provide an antagonistic effect on GAMS enhancement of the α3 subtypes of the GABA_{A}-receptor-chloride ionophore complexand thus acts acts as GAMS antagonists (GAMSA).Therefore, it is envisioned that the compounds may be used in the treatment of GAMS-related and/or steroid-induced disorders or diseases of the central nervous system (CNS) as well as diabetes. These and other objects are achieved in full, or at least in part, by aspects of the inventive concepts as disclosed herein.

Thus, in a third aspect of the present disclosure, there is provided a compound as disclosed herein, for use in prevention, alleviation and/or treatment of a steroid-related CNS disorder or disease, an autoimmune disease or of diabetes.

For the sake of clarity and avoidance of any doubt, as used herein in the context of therapeutic uses the terms “3α-ethynyl-3β-hydroxy-5α-androstan-17-methoxime”,“3α-ethyl-3β-hydroxy-5α-androstan-17-oxime”, “compound” and “compounds” are used interchangeably and are to be interpreted as encompassing 3α-ethynyl-3β-hydroxy-5α-androstan-17-methoxime and 3α-ethyl-3β-hydroxy-5α-androstan-17-oxime and any pharmaceutically acceptable salt, hydrate, prodrug and/or solvate thereof.

Similarly, for the sake of clarity and avoidance of any doubt, as used herein in the context of non-therapeutic (in other words cosmetic) uses the terms “3α-ethynyl-3β-hydroxy-5α-androstan-17-methoxime”,“3α-ethyl-3β-hydroxy-5α-androstan-17-oxime”, “compound” and “compounds” are used interchangeably and are to be interpreted as encompassing 3α-ethynyl-3β-hydroxy-5α-androstan-17-methoxime and 3α-ethyl-3β-hydroxy-5α-androstan-17-oxime and any cosmetically acceptable salt, hydrate, precursor and/or solvate thereof.

As used herein, the term “treatment” is used in the context of therapeutic treatment and relates to the treatment, such as causative or symptomatic treatment, of a disease or disorder, the alleviation of symptoms thereof and/or prevention of said disease or disorder. For example, it is envisioned that the obesity may be treated, alleviated or prevented by said treatment. For example, obesity may be a symptom of Prader-Willi's syndrome and may as such be treated, alleviated or prevented by said treatment.

According to the present disclosure, a GAMS is any steroid that positively modulates the GABA_{A} receptor. Typically, a positively modulating GAMS is a 3α-hydroxy-steroid. Non-limiting examples of such GAMS are 3α-hydroxy-5α/β-pregnan-20-one/ol, 3-α-hydroxy-5α/β-androstan-17-one/ol and tetrahydrodeoxycorticosterone (THDOC, 3α, 21-dihydroxy-5α-pregnan-20-one).

Non-limiting examples of symptoms and conditions associated with or caused by the direct action of 3α-hydroxy-5α/β-steroids are CNS disorders or diseases as follow: hyperphagia disorder; obesity; Prader-Willi's syndrome; polycystic ovarian syndrome; increased appetite disorder; obesity in diabetes; pathological food cravings; hypothalamic obesity; Cushing's syndrome; hyperphagia disorder associated with injury to the hypothalamus; alcoholism; substance use disorder; relapses into alcohol and/or substance use disorder; epilepsy; menstruation cycle dependent epilepsy; seizure disorder; worsening of Petit Mal epilepsy; memory disturbance; learning disturbance; menstrual cycle linked memory changes; stress related memory changes; stress related learning difficulties; hepatic encephalopathy; Down's syndrome; Alzheimer's disease; depression; stress related depression; premenstrual syndrome; premenstrual dysphoric disorder; menstrual cycle linked mood changes; negative mood such as as tension, irritability and depression; migraine; menstrual cycle linked migraine; stress linked migraine; hypersomnia and in particular stress related hypersomnia; sedation; idiopathic hypersomnia; sleep disorders; fatigue syndrome; burn-out syndrome; menstrual cycle linked sleep disorders; attention disorders; menstrual cycle linked difficulties in concentration; ADHD; mobility disorders; essential tremor; Tourette's syndrome; balance disturbances; disturbance of motor function; and clumsiness.

Also envisioned is that said compounds may be useful in treatment of fatty liver, insulin resistance, autoimmune diseases, and inflammatory disorders and symptoms. The present inventors envision that the compound disclosed herein may be useful in the treatment of said diseases or disorders.

In recent years, a large body of scientific publication has shown that GABA signaling is also involved in the immune system and implicated disease of the immune system and in inflammation. It has become evident that cells of the immune system may also produce GABA and express GABA_{A} receptors. These extra synaptic channels can be activated by low nano to micromolar GABA concentrations and such sub-micromolar GABA concentrations are present within the pancreas and in blood. The enzymes responsible for GABA synthesis and GABA_{A} receptors have been detected in all immunological competent cells e.g., T cells, macrophages, dendritic cells, macrophages monocytes and furthermore, GABAergic action is involved in the interactions between antigen presenting cells and T cells, between T and B cells in adaptive immune responses, or cytotoxic NK- and T-cell responses. The realization that extra synaptic GABA_{A} ion channels in immune cells can be fully activated by sub micromolar GABA concentrations makes GABA a potential effector molecule in many parts of the body including blood, pancreatic islets, cerebrospinal fluid and, of course, in the brain where the ambient GABA concentration is in the sub micromolar range. Research shows that mononuclear phagocytes and lymphocytes, dendritic cells, microglia, T cells and NK cells, express a GABAergic signaling machinery including membrane bound GABA_{A} receptors. Mounting evidence shows that GABA receptor signaling impacts important immune functions, such as cell migration, cytokine secretion, immune cell activation and cytotoxic responses (Bhandage, Barragan, 2021). Activation of GABA receptors on T cells and macrophages inhibits responses such as production of inflammatory cytokines. In T cells, GABA blocks the activation-induced calcium signal, and it also inhibits NF-κB activation. Furthermore, GABA clearly has an anti-inflammatory action, which is associated with inhibition of NF-κB activation. NF-κB activation is also blocked in pancreatic β cells, which may be of considerable therapeutic importance because this pathway induces apoptosis in these cells. (Prud'homme et al., 2015).

The absence of a presynaptic terminal defines these channels in the immune cells as extra synaptic-like channels existing in the brain. Physiologically this seems reasonable as the local concentration will be at nano or picomolar GABA concentrations close to immune cells in the blood when they enter the brain or the pancreatic islets. There are significant differences between the GABAergic stimulation depending on the GABA_{A} receptor subunit composition and therefore what subtypes are expressed in the immune cells. Properties like the affinity for GABA and the sensitivity to modulators' such as the benzodiazepines and steroids determine the effect of modulators (Lindquist and Birnir 2006; Olsen and Sieghart 2009). It is, therefore, of great importance if the modulators are specific to a certain subunit composition. GABA_{A} receptor subunit expression can be regulated with pharmacological agents (Uusi-Oukari, Korpi 2010).

Several studies have shown that GABA signalling may play a role in autoimmune disease and immune system related disease. For example, GABA ameliorates ongoing paralysis in experimental autoimmune encephalomyelitis (EAE) in mice models, by inhibiting onset of inflammation. GABA has also a role in rheumatoid arthritis and inflammatory responses to infection (Tian et al. 2011), autoimmune diseases like psoriasis, multiple sclerosis (Bath et al 2010), type I diabetes (Li et al 2017). Furthermore, the implication of GABA signaling in various autoimmune diseases, such as indicates a general role in inflammatory responses. In type 1 diabetes, GABA has been shown to have protective and stimulatory effects on β cells, but suppressive effects on the autoimmune response. (Prud'homme et al., 2015).

Thus, in one embodiment, there is provided said compound for use in the treatment of steroid related CNS disorder or disease selected from the group consisting of hyperphagia disorder; obesity; Prader-Willi's syndrome; polycystic ovarian syndrome; increased appetite disorder; obesity in diabetes; pathological food cravings; hypothalamic obesity; Cushing's syndrome; hyperphagia disorder associated with injury to the hypothalamus; alcoholims; substance use disorder; relapses into alcohol and/or substance use disorder; epilepsy; menstruation cycle dependent epilepsy; seizure disorder; worsening of Petit Mal epilepsy; memory disturbance; learning disturbance; menstrual cycle linked memory changes; stress related memory changes; stress related learning difficulties; hepatic encephalopathy; Down's syndrome; Alzheimer's disease; depression; stress related depression; premenstrual syndrome; premenstrual dysphoric disorder; menstrual cycle linked mood changes; negative mood such as as tension, irritability and depression; migraine; menstrual cycle linked migraine; stress linked migraine; hypersomnia and in particular stress related hypersomnia; sedation; idiopathic hypersomnia; sleep disorders; fatigue syndrome; burn-out syndrome; menstrual cycle linked sleep disorders; attention disorders; menstrual cycle linked difficulties in concentration; ADHD; mobility disorders; essential tremor; Tourette's syndrome; balance disturbances; disturbance of motor function; and clumsiness, such as the group consisting of hyperphagia disorder; obesity; Prader-Willi's syndrome; polycystic ovarian syndrome; increased appetite disorder; obesity in diabetes; pathological food cravings; hypothalamic obesity; Cushing's syndrome; hyperphagia disorder associated with injury to the hypothalamus; alcoholims; substance use disorder; relapses into alcohol and/or substance use disorder; epilepsy; menstruation cycle dependent epilepsy; seizure disorder; worsening of Petit Mal epilepsy; memory disturbance; learning disturbance; menstrual cycle linked memory changes; stress related memory changes; stress related learning difficulties; hepatic encephalopathy; Down's syndrome; Alzheimer's disease; depression; stress related depression; premenstrual syndrome; premenstrual dysphoric disorder; menstrual cycle linked mood changes; negative mood such as as tension, irritability and depression; migraine; menstrual cycle linked migraine; stress linked migraine; hypersomnia and in particular stress related hypersomnia; sedation; idiopathic hypersomnia; sleep disorders; fatigue syndrome; burn-out syndrome; menstrual cycle linked sleep disorders; attention disorders; menstrual cycle linked difficulties in concentration and ADHD; such as the group consisting of hyperphagia disorder; obesity; Prader-Willi's syndrome; polycystic ovarian syndrome; increased appetite disorder; obesity in diabetes; pathological food cravings; hypothalamic obesity; Cushing's syndrome; hyperphagia disorder associated with injury to the hypothalamus; alcoholims; substance use disorder; relapses into alcohol and/or substance use disorder; epilepsy; menstruation cycle dependent epilepsy; seizure disorder; worsening of Petit Mal epilepsy; memory disturbance; learning disturbance; menstrual cycle linked memory changes; stress related memory changes; stress related learning difficulties; hepatic encephalopathy; Down's syndrome; Alzheimer's disease; depression; stress related depression; premenstrual syndrome; premenstrual dysphoric disorder; menstrual cycle linked mood changes; negative mood such as as tension, irritability and depression; migraine; menstrual cycle linked migraine; stress linked migraine; hypersomnia and in particular stress related hypersomnia; sedation; idiopathic hypersomnia; sleep disorders; fatigue syndrome; burn-out syndrome and menstrual cycle linked sleep disorders, such as the group consisting of hyperphagia disorder; obesity; Prader-Willi's syndrome; polycystic ovarian syndrome; increased appetite disorder; obesity in diabetes; pathological food cravings; hypothalamic obesity; Cushing's syndrome; hyperphagia disorder associated with injury to the hypothalamus; alcoholims; substance use disorder; relapses into alcohol and/or substance use disorder; epilepsy; menstruation cycle dependent epilepsy; seizure disorder; worsening of Petit Mal epilepsy; memory disturbance; learning disturbance; menstrual cycle linked memory changes; stress related memory changes; stress related learning difficulties; hepatic encephalopathy; Down's syndrome; Alzheimer's disease; depression; stress related depression; premenstrual syndrome; premenstrual dysphoric disorder; menstrual cycle linked mood changes; negative mood such as as tension, irritability and depression; migraine; menstrual cycle linked migraine and stress linked migraine, such as the group consisting of hyperphagia disorder; obesity; Prader-Willi's syndrome; polycystic ovarian syndrome; increased appetite disorder; obesity in diabetes; pathological food cravings; hypothalamic obesity; Cushing's syndrome; hyperphagia disorder associated with injury to the hypothalamus; alcoholims; substance use disorder; relapses into alcohol and/or substance use disorder; epilepsy; menstruation cycle dependent epilepsy; seizure disorder; worsening of Petit Mal epilepsy; memory disturbance; learning disturbance; menstrual cycle linked memory changes; stress related memory changes; stress related learning difficulties; hepatic encephalopathy; Down's syndrome; Alzheimer's disease; depression; stress related depression; premenstrual syndrome; premenstrual dysphoric disorder; menstrual cycle linked mood changes; negative mood such as as tension, irritability and depression, such as the group consisting of hyperphagia disorder; obesity; Prader-Willi's syndrome; polycystic ovarian syndrome; increased appetite disorder; obesity in diabetes; pathological food cravings; hypothalamic obesity; Cushing's syndrome; hyperphagia disorder associated with injury to the hypothalamus; alcoholims; substance use disorder; relapses into alcohol and/or substance use disorder; epilepsy; menstruation cycle dependent epilepsy; seizure disorder; worsening of Petit Mal epilepsy; memory disturbance; learning disturbance; menstrual cycle linked memory changes; stress related memory changes; stress related learning difficulties; hepatic encephalopathy; Down's syndrome and Alzheimer's disease, such as the group consisting of hyperphagia disorder; obesity; Prader-Willi's syndrome; polycystic ovarian syndrome; increased appetite disorder; obesity in diabetes; pathological food cravings; hypothalamic obesity; Cushing's syndrome; hyperphagia disorder associated with injury to the hypothalamus; alcoholims; substance use disorder; relapses into alcohol and/or substance use disorder; epilepsy; menstruation cycle dependent epilepsy; seizure disorder and worsening of Petit Mal epilepsy, such as the group consisting of hyperphagia disorder; obesity; Prader-Willi's syndrome; polycystic ovarian syndrome; increased appetite disorder; obesity in diabetes; pathological food cravings; hypothalamic obesity; Cushing's syndrome; hyperphagia disorder associated with injury to the hypothalamus; alcoholims; substance use disorder; relapses into alcohol and/or substance use disorder.

In one particular embodiment, said CNS disorder or disease is selected from the group consisting of hyperphagia disorder; obesity; Prader-Willi's syndrome; polycystic ovarian syndrome; increased appetite disorder; obesity in diabetes; pathological food cravings; hypothalamic obesity; Cushing's syndrome and hyperphagia disorder associated with injury to the hypothalamus. In one particular embodiment, said CNS disorder or disease is selected from the group consisting of alcoholims; substance use disorder and relapses into alcohol and/or substance use disorder.

Table 1 lists all receptor subtypes of the GABA_{A} receptor.

TABLE 1

| Isoform | Synaptic/Extrasynaptic | Anatomical location |
|---|---|---|
| α1β3γ2S | Both | Widespread |
| α2β3γ2S | Both | Widespread |
| α3β3γ2S | Both | Reticular thalamic |
| | | nucleus |
| α4β3γ2S | Both | Thalamic relay cells |
| α5β3γ2S | Both | Hippocampal pyramidal |
| | | cells |
| α6β3γ2S | Both | Cerebellar granule cells |
| α1β2γ2S | Both | Widespread, most |
| | | abundant |
| α4β3δ | Extrasynaptic | Thalamic relay cells |
| α6β3δ | Extrasynaptic | Cerebellar granule cells |
| α1β2 | Extrasynaptic | Widespread |
| α1β3 | Extrasynaptic | Thalamus, hypothalamus |
| α1β2δ | Extrasynaptic | Hippocampus |
| α4β2δ | Extrasynaptic | Hippocampus |
| α3β3θ | Extrasynaptic | Hypothalamus |
| α3β3ε | Extrasynaptic | Hypothalamus |

GAMSAs with specificity to an α3-subtype GABA_{A} receptor were unknown up to date. As shown in Table 1, there are three known GABA_{A} receptor α3 subtype, namely α3β3γ2, α3β3θ and α3β3ε. The present inventors have found that when acting on the GABA_{A} receptor α3 subtype, the compound disclosed herein are a partial antagonists to GABA and a full antagonist to 3α-hydroxy-pregnan/androstan-steroids. Exposure to a 3α-hydroxy-pregnan/androstane-steroid increases the chloride flux through the human GABA_{A} receptor of any subtype but the compounds of the present disclosure inhibit the chloride flux through the human GABA_{A} receptor α3 subtype. The effect is induced by GABA or induced by a 3α-hydroxy steroid combined with GABA. This has been tested in recombinantly expressed human embryonic kidney cells (HEK-cells) expressing the GABA_{A} receptor α3β2γ2 subtype, see the appended Examples (Example 1).

Thus, in one embodiment, said α3 subtype of the GABA_{A} receptor is α3β3γ2.

The inhibitory effect on GABAs and/or agonistic effect (a GAMS's effect) is surprising since it was known to a person of skill in the art that similar compounds (having a 3β-hydroxy configuration) have no antagonistic or agonistic effect on GABA's own ability to open the receptor for flux of chloride ions. In prior art, similar compounds have either had no effect or have enhanced the effect of GABA and thus increased the chloride flux through the receptor (see for example U.S. Pat. No. 5,925,630).

Interestingly, 3α-ethynyl-3β-hydroxy-5α-androstan-17-methoxime and 3α-ethyl-3β-hydroxy-5α-androstan-17-oxime disclosed herein efficiently antagonize the GABA_{A} receptor modulation effect of 3α-hydroxy-5α/β-pregnan/androstane-steroids on the α3β3γ2 GABA_{A} receptor subtype. As such, it is envisioned that it is possible to selectively block the action of 3α-hydroxy-5α/β-pregnan/androstane-steroids on the α3β3γ2 GABA_{A} receptor by simultaneous administration of 3α-ethynyl-3β-hydroxy-5α-androstan-17-methoxime and/or 3α-ethyl-3β-hydroxy-5α-androstan-17-oxime in pharmaceutically and physiologically acceptable amounts. Thereby, it is plausible that major advantages are achieved when the compound of the present invention is administrated while elevated doses of GAMS (either endogenous or administered) or increased sensitivity to GAMS are present in the body or CNS of a subject.

Without being bound by theory, it is envisioned that the selectively allows for administering a high dose when therapeutically motivated without the patient experiencing adverse side effects and/or allows for administering a low dose, for example during long term treatment, and still achieve a desired therapeutic outcome due to said selectivity.

Beneficially, this blocking may be achieved at pharmacologically and physiologically suitable concentrations as discussed in detail below.

Thus, in one embodiment, there is provided the compound as disclosed herein for use in antagonizing GABA signaling via the α3β3γ2 GABA_{A} receptor subtype.

In one embodiment of third aspect of the present disclosure, said CNS disorder or disease, autoimmune disease, or diabetes is associated with an α3 subtype of the GABA_{A} receptor, such as the α3β2γ2 subtype of the GABA_{A} receptor. In one embodiment of the present disclosure, there is provided the compound for use a disclosed herein, wherein steroid-related CNS disorder or disease is selected from the group consisting of hyperphagia disorder; obesity; Prader-Willi's syndrome; polycystic ovarian syndrome; increased appetite disorder; obesity in diabetes; pathological food cravings; hypothalamic obesity; Cushing's syndrome; hyperphagia disorder associated with injury to the hypothalamus; alcoholims; substance use disorder; relapses into alcohol and/or substance use disorder, such as group consisting of hyperphagia disorder; obesity; Prader-Willi's syndrome; polycystic ovarian syndrome; increased appetite disorder; obesity in diabetes; pathological food cravings; hypothalamic obesity; Cushing's syndrome and hyperphagia disorder associated with injury to the hypothalamus.

In one embodiment said disease or disorder hyperphagia disorder; obesity; Prader-Willi's syndrome; polycystic ovarian syndrome; increased appetite disorder; obesity in diabetes; pathological food cravings; hypothalamic obesity; Cushing's syndrome; hyperphagia disorder associated with injury to the hypothalamus and diabetes; such as
the group consisting of hyperphagia disorder, obesity, Prader-Willi's syndrome, polycystic ovarian syndrome, and diabetes.

In one embodiment, said disease is selected from the group consisting of obesity, hyperphagia disorder and Prader-Willi's syndrome; or the group consisting of obesity, hyperphagia disorder and polycystic ovarian syndrome; or the group consisting of obesity, hyperphagia disorder and diabetes. In one embodiment, said disease is selected from obesity and hyperphagia disorder. In one embodiment, said disease is selected from obesity and diabetes. In one embodiment, said diabetes is diabetes type II.

Obese children as well as obese adults have been reported to have high blood concentrations of GAMS. Thus, in one embodiment, said disorder or disease is obesity.

Said obesity may be hypothalamic obesity. Hypothalamic obesity refers to obesity that is caused by physical or inborn damage to the hypothalamus (Rose et al., 2018). The hypothalamus is part of the brain that makes hormones that control specific body functions such as sleep, body temperature, and hunger. It also makes hormones that control other organs in the body, especially the pituitary gland. The symptoms of hypothalamic obesity vary by the cause and include uncontrollable hunger, rapid, excessive weight gain, and a low metabolic rate. If the pituitary gland is involved, symptoms may include small underdeveloped testes in males and delayed puberty. This condition most often occurs because of injury to the hypothalamus due to a tumor, swelling in the brain, brain surgery, or head trauma. The diagnosis is made by physical examination and review of the symptoms. There is no cure for hypothalamic obesity. At present, treatment involves a combination of surgery, medications, and nutritional and lifestyle counseling. The long-term outlook for people with this condition is dependent on weight loss and management.

Hyperphagia disorder relates to an abnormally great desire for food and/or excessive eating. Non-limiting examples of hyperphagia disorder comprise binge eating disorder, hyperphagia disorder associated with injury to the hypothalamus, and Prader-Willi's syndrome. A patient suffering from binge eating disorder suffers from recurrent episodes of eating large quantities of food and a feeling of loss of control. In one embodiment, said disease or disorder is a hyperphagia disorder, such as hyperphagia disorder resulting in overweight and/or obesity. In one embodiment, said hyperphagia disorder is binge eating disorder or hyperphagia disorder associated with injury to the hypothalamus.

For maintaining health, energy intake should normally be in balance with energy expenditure. For example, in the group of moderately active adults at age 66 or older, men are advised to eat about 2200 kilo calories per day and women are advised to eat about 1800 kilo calories per day. To avoid unhealthy weight gain, total fat should not exceed 30% of total energy intake. Intake of saturated fats should be less than 10% of total energy intake, and intake of trans-fats less than 1% of total energy intake, with a shift in fat consumption away from saturated fats and trans-fats to unsaturated fats, and towards the goal of eliminating industrially-produced trans-fats. In one embodiment, said hyperphagia disorder comprises eating at least 105% of the individual's energy expenditure, such as 110% of an individual's energy expenditure, such as 115% of an individual's energy expenditure, such as 120% of an individual's energy expenditure, such as 125% of an individual's energy expenditure, such as 130% of an individual's energy expenditure, such as 135% of an individual's energy expenditure, such as 140% of an individual's energy expenditure, such as 145% of an individual's energy expenditure, such as 150% of an individual's energy expenditure, such as 155% of an individual's energy expenditure, such as 160% of an individual's energy expenditure, such as 165% of an individual's energy expenditure, such as 170% of an individual's energy expenditure, such as 175% of an individual's energy expenditure, such as 180% of an individual's energy expenditure, such as 185% of an individual's energy expenditure, such as 190% of an individual's energy expenditure, such as 195% of an individual's energy expenditure, such as 200% of an individual's energy expenditure, such as 210% of an individual's energy expenditure, such as 220% of an individual's energy expenditure, such as 230% of an individual's energy expenditure, such as 240% of an individual's energy expenditure, such as 250% of an individual's energy expenditure, such as 260% of an individual's energy expenditure, such as 270% of an individual's energy expenditure, such as 280% of an individual's energy expenditure, such as 290% of an individual's energy expenditure, such as 300% of an individual's energy expenditure.

People suffering from Prader-Willi's syndrome have problems with hyperphagia disorder from young age and often become overweight or even obese already during the teenage years. These subjects typically exhibit an over expression of GABA_{A} receptor subunits that are highly sensitive to GAMS. Thus, in one embodiment, said disease or disorder is Prader-Willi's syndrome. In one embodiment, said disease is Prader-Willi's syndrome resulting in overweight and/or obesity. In other words, overweight and/or obesity associated with Prader-Willi's syndrome may be treated.

Women with PolyCystic Ovarian Syndrome (PCOS) have also been reported to exhibit high levels of GAMS. In one embodiment, said disease is polycystic ovarian syndrome. Over 60% of the women with this disorder are obese or overweight. Therefore, in one embodiment, said disease or disorder is polycystic ovarian syndrome, such as polycystic ovarian syndrome resulting in overweight and/or obesity. In another embodiment, said disease is obesity associated with to polycystic ovarian syndrome. In other words, overweight and/or obesity associated with polycystic ovarian syndrome may be treated.

Modulators of the GABA_{A} receptor can affect the insulin production, immunological functions and insulin resistance in diabetes type II (Tian et al., Prud'homme et al.) Thus, in a related embodiment, said disease is obesity associated with diabetes. In one related embodiment, said diabetes is diabetes type II.

Additionally, obesity and/or hyperphagia disorder may increase the risk for developing type II diabetes. Obesity and diabetes type II are a common comorbidity. In one embodiment, said diabetes is associated with overweight and/or obesity. Obesity and diabetes type II are a common comorbidity. In one embodiment, said diabetes is associated with overweight and/or obesity.

In yet another embodiment, said disease is an autoimmune disease. In one embodiment, said autoimmune disease may be diabetes type I. Obesity in diabetes type I is a disadvantage since it may be harder to control the diabetes and maintain insulin at healthy levels. For example, if it is hard to control the intake of food it will be hard to control the dosing of insulin. This may lead to an elevated probability to reach a hypoglycemic state.

The GABA-system is involved in diseases such as alcoholism and drug abuse. Modulators of the GABA_{A} receptor can affect the urge of abusing alcohol and/or substances. In this context, the substance may is any substance whose ingestion can result in a euphoric (“high”) feeling. In one embodiment, wherein said CNS disorder or disease is alcoholism, substance use disorder or relapse into alcoholism and/or substance abuse disorder. In one embodiment said disease or disorder is alcoholism. In one embodiment, said disease or disorder is substance use disorder.

As used herein the term, “drug use disorder” or “substance use disorder”, refers to a a disease that affects a person's brain and behavior and leads to an inability to control the use of a legal or illegal drug or medication. Substances such as marijuana and nicotine also are considered drugs.

As used herein, the terms “alcoholism” and “alcoholism use disorder” are used interchangably.

The GABA system may be involved in the pathophysiology of obsessive-compulsive disorder. In one embodiment, said disease is obsessive-compulsive disorder.

Without being bound by theory, it is envisioned that the compounds as disclosed herein may be useful to selectively block the action of 3α-hydroxy-5α/β-pregnan/androstane-steroids on the α3β3γ2 GABA_{A} receptor. As discussed above, it is plausible that major advantages are achieved when the one or both compounds of the present invention are administrated while elevated doses of GAMS (either endogenous or administered) or increased sensitivity to GAMS are present in the body or CNS of a subject. Thus, it is envisionend that the compound may be useful in prevention, alleviation and/or treatment of a condition caused by exposure to at least one 3α-hydroxy-steroid endogenous or exogenous. In one particular embodiment, the are provided the compounds as disclosed herein for use in prevention, alleviation and/or treatment of a side effect caused by an anti-inflammatory steroid, postmenopausal therapy, and/or an oral contraceptive. Said side effect may be caused by elevated levels of 3α-hydroxy-5α/β-pregnan/androstane-steroids.

It is envisioned that the administration of 3α-ethynyl-3β-hydroxy-5α-androstan-17-methoxime, 3α-ethyl-3β-hydroxy-5α-androstan-17-oxime or a pharmaceutically acceptable salt, hydrate, prodrug or solvate thereof leads to a decrease of bodyweight. For example, the decrease in bodyweight may be seen after 1 to 100 days, such as 2 to 20 days, such as after 3 to 15 days, such as after 5 to 10 days. Thus, in one embodiment said treatment results in a decrease in bodyweight after 1 to 100 days, such as 2 to 20 days, such as after 3 to 15 days, such as after 5 to 10 days, of treatment. In one particular embodiment, said treatment results in a decrease of daily calory intake by at least about 10%, such as at least 15%, such as at least 20%, such as at least 25%, such as at least 30%, such as at least 35%, such as at least 40%, such as at least 45%, such as at least 50%.

Naturally occurring steroids are subject to intense metabolism and are typically not suitable for oral administration as they quickly become degraded without sufficient time to exert its desired pharmacological effect.

The present invention provides the use of a synthetic steroid with high water solubility compared to other steroids known to effect GABA signaling. In particular, the presence of an ethynyl moiety in position 3 (3α) in the compound according to Formula 1 has been shown to be able to prolong the half-life of a steroidal compound within the body of a mammal. Without being bound by theory, the effect may be through preventing metabolic oxidations or degradation in said body.

According to the present disclosure, 3α-ethynyl-3β-hydroxy-5α-androstan-17-methoxime and 3α-ethyl-3β-hydroxy-5α-androstan-17-oxime may be administered by one of the following routes of administration: intravenously, nasally, per rectum, intravaginally, percutaneously, subcutaneously, transdermally, intramuscularly, or orally. In one embodiment, said 3α-ethynyl-3β-hydroxy-5α-androstan-17-methoxime and/or 3α-ethyl-3β-hydroxy-5α-androstan-17-oxime is administered by a route of administration selected from the group consisting of intravenous, nasal, per rectum, intravaginal, percutaneous, subcutaneous, transdermal, intramuscular and oral administration; such as the group consisting of nasal, per rectum, intravaginal, subcutaneous, transdermal, intramuscular and oral administration; such as the group consisting of nasal, subcutaneous, transdermal and oral administration. In one embodiment, said route of administration is selected from the group consisting of nasal, per rectum, intravaginal, percutaneous, subcutaneous, transdermal, intramuscular and oral administration; such as the group consisting of nasal, percutaneous, subcutaneous, transdermal and oral administration. In one embodiment, said route of administration is selected from the group consisting of nasal, oral and subcutaneous administration or nasal, oral and percutaneous administration. According to one embodiment, 3α-ethynyl-3β-hydroxy-5α-androstan-17-methoxime and/or 3α-ethyl-3β-hydroxy-5α-androstan-17-oxime is administered intravenously.

It is considered that ways of administration which are simple and easy for the patient, causing minimal discomfort if any, are desirable and also increase patient compliance with treatment. Nasal administration is envisioned as a promising administration alterative, as it offers the benefits of ease and the possibility of self-administration by a patient. Similarly, oral administration is envisioned as a promising administration alternative also allowing for self-administration without assistance of others. Thus, according to another embodiment, 3α-ethynyl-3β-hydroxy-5α-androstan-17-methoxime and/or 3α-ethyl-3β-hydroxy-5α-androstan-17-oxime is administered nasally. In one embodiment, 3α-ethynyl-3β-hydroxy-5α-androstan-17-methoxime and/or 3α-ethyl-3β-hydroxy-5α-androstan-17-oxime is administered orally.

Self-administration has the advantage of allowing a patient to adjust the dose or the frequency of medication either according to a subjective evaluation of their condition or according to a schedule prescribed by a treating physician. The term “schedule prescribed by the treating physician” includes the alternative where a patient makes a subjective evaluation of his/her condition, either unaided or aided by a questionnaire or a range or scale, or using an algorithm or a computer program, indicating the suitable next dose.

Percutaneous administration, using 3α-ethynyl-3β-hydroxy-5α-androstan-17-methoxime and/or 3α-ethyl-3β-hydroxy-5α-androstan-17-oxime formulated as a cream, a gel, and an ointment or in the form of slow-release adhesive medicine patches, is another possible form of administration, similarly suitable for self-medication. The advantages of self-administration listed above apply also to percutaneous administration, with the added advantage that the administration can easily be interrupted if desired or necessary, e.g. by removing the medicine patch.

It is also possible that administration is via a depot formulation, which releases an effective amount of the therapeutically active compound as disclosed herein, over a period of time. The skilled person will appreciate that the depot formulation may be adapted to deliver the desired effective dose as prescribed by a treating physician. A depot formulation may be a subcutaneous depot formulation. Thus, in one embodiment, said administration via a depot formulation, such as a subcutaneous depot formulation.

In any of these or other routes of administration, the formulation of the composition may be adapted or adjusted according to normal pharmacological procedures, comprising the effective pharmaceutical in a chemical form, suitable for the chosen route, together with suitable excipients, such as adjuvants, carriers, diluents and vehicles, conventionally used and well-known to a person skilled in the art.

Conventionally used adjuvants and vehicles for oral administration are for example fillers or suspending agents like titanium dioxide, lactose anhydride, silica, silica colloidalis, methylcellulose, magnesium stearate, microcrystalline cellulose and the like. As used herein, the term “adjuvant” relates to a compound which potentiates the effect of the pharmaceutically active compound.

Conventionally used excipients for intravenous administration are for example sterile water for injections (WFI), sterile buffers (for example buffering the solution to pH 7.4) albumin solution, lipid solutions, cyclodextrin and variants thereof, and the like.

Conventionally used excipients for subcutaneous administration are for example sterile water for injections (WFI), sterile buffers (for example buffering the solution to pH 7.4) lipid solutions, cyclodextrins and the like.

Conventionally used excipients for subcutaneous administration via a subcutaneous delivery system, such as a subcutaneous rod, are for example sterile water for injections (WFI), sterile buffers (for example buffering the solution to pH 7.4) lipid solutions, cyclodextrins and the like.

Conventionally used excipients for transdermal and/or subcutanous administration are for example vaseline, liquid paraffin, glycerol, water, MCT oil, sesame oil and the like.

The skilled person will appreciate that the suitable dose will naturally vary depending on the mode of administration, the particular condition to be treated or the effect desired, gender, age, weight and health of the patient, as well as possibly other factors, evaluated by the treating physician. According to the present disclosure, 3α-ethynyl-3β-hydroxy-5α-androstan-17-methoxime and/or 3α-ethyl-3β-hydroxy-5α-androstan-17-oxime may be administered intravenously, a suitable dose may be that ranging from about 0.1 to about 300 mg per kg body weight. Preliminary studies in animals indicate that a preferred dose interval for intravenous administration is from about 20 to about 100 mg per kg body weight. In one embodiment, said compound is administrated in an effective dose in the range of from about 0.1 to about 300 mg per kg body weight, such as in a dose in the range of from about 0.2 to about 200 mg per kg body weight, such as in a dose in the range of from about 0.3 to about 150 mg, such as in a dose in the range of from about 0.4 to about 150 mg per kg bodyweight, such as in a dose in the range of from about 0.5 to about 120 mg per kg bodyweight, such as in a dose in the range of from about 1 to about 100 mg per kg body weight, such as in a dose in the range of from about 1 to about 50 mg per kg body weight, such as in a dose in the range of from about 1 to about 5 mg per kg body weight, such as about 1 mg per kg body weight.

It is envisioned that a therapeutically effective concentration of 3α-ethynyl-3β-hydroxy-5α-androstan-17-methoxime and/or 3α-ethyl-3β-hydroxy-5α-androstan-17-oxime may be in the range of from about 10 mg/day to about 30 g/day, such as of about 20 mg/day to about 20 g/day. In one embodiment, said compound is administrated in a dose in the range of from about 30 mg to about 15 g/day, such as in an effective dose in the range of from about 40 mg/day to about 15 g day, such as in a dose in the range of from about 50 mg/day to about 12 g/day, such as in a dose in the range of from about 100 mg/day to about 10 g/day, such as in a dose in the range of from about 100 mg/day to about 5 g/day, such as in a dose in the range of from about 100 mg/day to about 500 mg/day. In one embodiment, said compound is administrated in a dose in the range of from about 20 mg/day to about 60 g/day, such as in a dose in the range of from about 40 mg/day to about 40 g/day, such as in a dose in the range of from about 60 mg/day to about 30 g/day, such as in a dose in the range of from about 80 mg/day to about 30 g/day, such as in a dose in the range of from about 100 mg/day to about 24 g/day, such as in a dose in the range of from about 200 mg/day to about 20 g/day, such as in a dose in the range of from about 200 mg/day to about 10 g/day, such as in a dose in the range of from about 200 mg/day to about 1 g/day.

Said above mentioned doses are to be understood to refer to therapeutically effective doses.

As apparent to a person of skill in the art, 3α-ethynyl-3β-hydroxy-5α-androstan-17-methoxime and/or 3α-ethyl-3β-hydroxy-5α-androstan-17-oxime may be administered at one or more occasions per day. In one embodiment, 3α-ethynyl-3β-hydroxy-5α-androstan-17-methoxime and/or 3α-ethyl-3β-hydroxy-5α-androstan-17-oxime is administered once per day. In another embodiment, 3α-ethynyl-3β-hydroxy-5α-androstan-17-methoxime and/or 3α-ethyl-3β-hydroxy-5α-androstan-17-oxime is administered twice per day, or even three or four times per day. It may be suitable that the administration is in connection with meals, such as the three main meals of the day (for example breakfast, lunch and dinner or other meal schedule which is relevant and suitable for the patient, for example a more frequent meal schedule). It may also be suitable that the compound of the invention is administrated less frequently, such as every second day, or every third day, or even once every week. In embodiments where the compound of the present disclosure is administrated as a subcutaneous implant or depot, the administration of the compound is a continuous process under diffusion. Such implant or depot may be inserted to a patient and may last for at least one month, such as for at least six months, such as for at least one year, such at least for two years, such as for at least three years, such as for at least four years, such as for at least five years, such as for at least six years. In one embodiment, wherein the compound is administrated as a subcutaneous implant or depot, it may be beneficial that the daily dose of is constant over a desired therapeutic period. The daily dose administered may be as discussed in the section above and is not repeated here for the sake of brevity.

In one embodiment, said compound as disclosed herein or compound for use as disclosed herein provides an antagonistic effect on the effect of γ-aminobutyric acid (GABA) and/or the effect of any GABA_{A} receptor modulating steroids (GAMS) on a GABA_{A} receptor α3 subtype, such as on the GABA_{A} receptor α3β2γ2 subtype. In one embodiment, said compound or compound for use as disclosed herein provides an antagonistic effect on the effect of γ-aminobutyric acid (GABA) on said GABA_{A} receptor α3 subtype. In another embodiment, said compound or compound for use as disclosed herein provides an antagonistic effect on the effect of any GABA_{A} receptor modulating steroids (GAMS) on said GABA_{A} receptor α3 subtype, for example on the effect of THDOC.

As described above, GABA is the main inhibitory neurotransmitter in the central nervous system, and in the rest of the body. Therefore, it is envisioned, without being bound by theory, that a 100% antagonistic effect of GABA may give rise to serious side effects. As such, said antagonistic effect achieved by the compound as disclosed herein is preferably a partial antagonistic effect. Thus, in one embodiment the antagonistic effect of said compound on GABA signaling via the α3 subtype GABA_{A} receptor is least 1%, such as at least 2%, such as at least 3%, such as at least 4%, such as at least 5%, such as at least 10%, such as at least 15%, such as at least 20%, such as at least 25%, such as at least 30%, such as at least 35%, such as at least 40%, such as at least 45%, such as at about 50%.

Preferably, said partial antagonistic effect is at most 80%, such as at most 75%, such as at most 70%, such as at most 65%, such as at most 60%, such as at most 55%, such as at most 50%.

Thus, in one embodiment said compound antagonizes GABA signaling via the α3 subtype GABA_{A} receptor by at most 80%, such as at most 75%, such as at most 70%, such as at most 65%, such as at most 60%, such as at most 55%, such as at most 50%.

For clarity, an antagonistic effect of 70% is to be interpreted as that 30% activity still remains.

The skilled person will appreciate that the compound for use as disclosed herein may be administered at a dose which achieves said partial antagonistic effect.

As discussed above, said compound provides an antagonistic effect on the effect of γ-aminobutyric acid (GABA) and/or any GABA_{A} receptor modulating steroids (GAMS) on an α3 subtype of the GABA_{A} receptor, such as the α3β2γ2 subtype of the GABA_{A} receptor. In one embodiment, said compound for use as disclosed herein further provides an antagonistic effect on the effect of γ-aminobutyric acid (GABA) and/or any GABA_{A} receptor modulating steroids (GAMS) on the GABA_{A} receptor α1, α2, α4 and/or α5 subtype.

As discussed above, said compound provides an antagonistic effect on the effect of γ-aminobutyric acid (GABA) and/or any GABA_{A} receptor modulating steroids (GAMS) on an α3 subtype of the GABA_{A} receptor, such as the α3β2γ2 subtype of the GABA_{A} receptor. In one embodiment, said compound for use as disclosed herein further provides an antagonistic effect on the effect of γ-aminobutyric acid (GABA) and/or any GABA_{A} receptor modulating steroids (GAMS) on the GABA_{A} receptor α1, α2, α4 and/or α5 subtype, such as the α2, α4 and/or α5 subtype. In one embodiment, said compound further provides an antagonistic effect on the effect of γ-aminobutyric acid (GABA) and/or any GABA_{A} receptor modulating steroids (GAMS) on the GABA_{A} receptor α3 and α1 subtypes. In one embodiment, said compound further provides an antagonistic effect on the effect of γ-aminobutyric acid (GABA) and/or any GABA_{A} receptor modulating steroids (GAMS) on the GABA_{A} receptor α3 and α2 subtypes. In one embodiment, said compound further provides an antagonistic effect on the effect of γ-aminobutyric acid (GABA) and/or any GABA_{A} receptor modulating steroids (GAMS) on the GABA_{A} receptor α3 and α4 subtypes. In one embodiment, said compound further provides an antagonistic effect on the effect of γ-aminobutyric acid (GABA) and/or any GABA_{A} receptor modulating steroids (GAMS) on the GABA_{A} receptor α3 and α5 subtypes. In one embodiment, said compound further provides an antagonistic effect on the effect of γ-aminobutyric acid (GABA) and/or any GABA_{A} receptor modulating steroids (GAMS) on the GABA_{A} receptor α3, α2 and α1 subtypes. In one embodiment, said compound further provides an antagonistic effect on the effect of γ-aminobutyric acid (GABA) and/or any GABA_{A} receptor modulating steroids (GAMS) on the GABA_{A} receptor α3, α2 and α4 subtypes. In one embodiment, said compound further provides an antagonistic effect on the effect of γ-aminobutyric acid (GABA) and/or any GABA_{A} receptor modulating steroids (GAMS) on the GABA_{A} receptor α3, α1 and α5 subtypes. In one embodiment, said compound further provides an antagonistic effect on the effect of γ-aminobutyric acid (GABA) and/or any GABA_{A} receptor modulating steroids (GAMS) on the GABA_{A} receptor α3, α2 and α4 subtypes. In one embodiment, said compound further provides an antagonistic effect on the effect of γ-aminobutyric acid (GABA) and/or any GABA_{A} receptor modulating steroids (GAMS) on the GABA_{A} receptor α3, α2 and α5 subtypes. In one embodiment, said compound further provides an antagonistic effect on the effect of γ-aminobutyric acid (GABA) and/or any GABA_{A} receptor modulating steroids (GAMS) on the GABA_{A} receptor α3, α4 and α5 subtypes. In one embodiment, said compound further provides an antagonistic effect on the effect of γ-aminobutyric acid (GABA) and/or any GABA_{A} receptor modulating steroids (GAMS) on the GABA_{A} receptor α1, α2, α3 and α4 subtypes. In one embodiment, said compound further provides an antagonistic effect on the effect of γ-aminobutyric acid (GABA) and/or any GABA_{A} receptor modulating steroids (GAMS) on the GABA_{A} receptor α1, α2, α3 and α5 subtypes. In one embodiment, said compound further provides an antagonistic effect on the effect of γ-aminobutyric acid (GABA) and/or any GABA_{A} receptor modulating steroids (GAMS) on the GABA_{A} receptor α1, α3, α4 and α5 subtypes.

As discussed above, said compound provides an antagonistic effect on the effect of γ-aminobutyric acid (GABA) on an α3 subtype of the GABA_{A} receptor, such as the α3β2γ2 subtype of the GABA_{A} receptor.

In one embodiment, said compound for use as disclosed herein further provides an antagonistic effect on the effect of γ-aminobutyric acid (GABA) on the GABA_{A} receptor α1, α2, α4 and/or α5 subtype, such as the α2, α4 and/or α5 subtype. In one embodiment, said compound further provides an antagonistic effect on the effect of γ-aminobutyric acid (GABA) on the GABA_{A} receptor α3 and α1 subtypes. In one embodiment, said compound further provides an antagonistic effect on the effect of γ-aminobutyric acid (GABA) on the GABA_{A} receptor α3 and α2 subtypes. In one embodiment, said compound further provides an antagonistic effect on the effect of γ-aminobutyric acid (GABA) on the GABA_{A} receptor α3 and α4 subtypes. In one embodiment, said compound further provides an antagonistic effect on the effect of γ-aminobutyric acid (GABA) on the GABA_{A} receptor α3 and α5 subtypes. In one embodiment, said compound further provides an antagonistic effect on the effect of γ-aminobutyric acid (GABA) on the GABA_{A} receptor α3, α2 and α1 subtypes. In one embodiment, said compound further provides an antagonistic effect on the effect of γ-aminobutyric acid (GABA) on the GABA_{A} receptor α3, α2 and α4 subtypes. In one embodiment, said compound further provides an antagonistic effect on the effect of γ-aminobutyric acid (GABA) on the GABA_{A} receptor α3, α1 and α5 subtypes. In one embodiment, said compound further provides an antagonistic effect on the effect of γ-aminobutyric acid (GABA) on the GABA_{A} receptor α3, α2 and α4 subtypes. In one embodiment, said compound further provides an antagonistic effect on the effect of γ-aminobutyric acid (GABA) on the GABA_{A} receptor α3, α2 and α5 subtypes. In one embodiment, said compound further provides an antagonistic effect on the effect of γ-aminobutyric acid (GABA) on the GABA_{A} receptor α3, α4 and α5 subtypes. In one embodiment, said compound further provides an antagonistic effect on the effect of γ-aminobutyric acid (GABA) on the GABA_{A} receptor α1, α2, α3 and α4 subtypes. In one embodiment, said compound further provides an antagonistic effect on the effect of γ-aminobutyric acid (GABA) on the GABA_{A} receptor α1, α2, α3 and α5 subtypes. In one embodiment, said compound further provides an antagonistic effect on the effect of γ-aminobutyric acid (GABA) on the GABA_{A} receptor α1, α3, α4 and α5 subtypes.

The level or degree of the antagonistic effect of said compound on GABA signaling via the α1, α2, α4 and/or α5 subtype(s) of the GABA_{A} receptor may differ from the same antagonistic effect via the α3 subtype of the GABA_{A} receptor, such as the α3β2γ2 subtype of the GABA_{A} receptor. Thus, in one embodiment the antagonistic effect of said compound on GABA signaling via the α1, α2, α4 and/or α5 subtype GABA_{A} receptor is within the range of 1-30%, such as within the range of 2-25%, such as within the range of 3-22%, such as within the range of 4-20%, such as within the range of 5-15%, such as within the range of 7-13%, such as within the range of 8-10%. It will be understood that said ranges are equally relevant for any one of said further provided antagonistic effects on the effect of γ-aminobutyric acid (GABA) and/or any GABA_{A} receptor modulating steroids (GAMS) on individual or subsets of GABA_{A} receptor subtypes (see above).

The present inventors have shown that 3α-ethynyl-3β-hydroxy-5α-androstan-17-methoxime and 3α-ethyl-3β-hydroxy-5α-androstan-17-oxime provide unique effect on the different GABA_{A} receptor subtypes, compared to other similar known steroid ligands for this receptor. Furthermore, 3α-ethynyl-3β-hydroxy-5α-androstan-17-methoxime and 3α-ethyl-3β-hydroxy-5α-androstan-17-oxime also provide a unique effect on GAMS effect on GABA_{A} receptor subtypes, such as GAMS effect on the α3 subtype.

It will be appreciated that the GAMS antagonist described herein may form part of a pharmaceutical composition. Thus, in a fourth aspect, there is provided a pharmaceutical composition comprising 3α-ethynyl-3β-hydroxy-5α-androstan-17-methoxime and/or 3α-ethyl-3β-hydroxy-5α-androstan-17-oxime as described herein, and at least one pharmaceutically acceptable excipient(s).

As used herein, the term “excipient” encompasses adjuvants, carriers, diluents, and vehicles. The skilled person appreciates that any adjuvants, carriers, diluents and vehicles mentioned in connection with the first aspect as disclosed herein are suitable in said pharmaceutical composition and it is withing the knowledge of the skilled person to make the appropriate choice thereof. The skilled person appreciates that the pharmaceutical composition may be adapted to be suitable for the selected administration route as well as desired administered dose. Relevant doses and administration routes are disclosed in connection to the third aspect above.

Non-limiting examples of suitable carriers are cyclodextrin, sterile water for injections (WFI), sterile buffers (for example buffering the solution to pH 7.4) albumin solution, lipid solutions, cyclodextrin variants and the like.

In a related fifth aspect, there is provided a method of treatment, alleviation and/or prevention of a steroid-related CNS disorder or disease, an autoimmune disease, or of diabetes, comprising the step of administering a pharmaceutically effective amount of comprising administering a pharmaceutically effective amount of compound selected from the group consisting of
3α-ethynyl-3β-hydroxy-5α-androstan-17-methoxime as shown in Formula 1

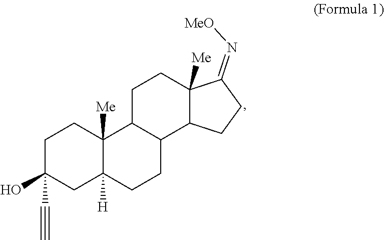 [SMILES: C#C[C@]1(O)CC[C@]2(C)C3CC[C@]4(C)/C(=N\OC)CCC4C3CC[C@H]2C1]

and
3α-ethyl-3β-hydroxy-5α-androstan-17-oxime as shown in Formula 2

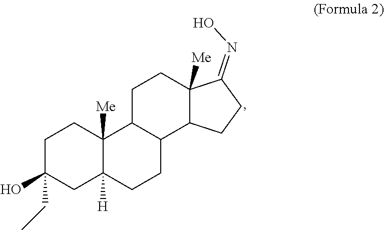 [SMILES: CC[C@]1(O)CC[C@]2(C)C3CC[C@]4(C)/C(=N\O)CCC4C3CC[C@H]2C1]

or
a pharmaceutically acceptable salt, hydrate, prodrug or solvate thereof, to a patient in need thereof as disclosed herein or a pharmaceutical composition as disclosed herein, to a patient in need thereof. It will be appreciated that the embodiments disclosed in connection with the third aspect, including embodiments related to dose, frequency of administration and route of administration, are equally relevant for this fifth aspect as for aspects three and four and are not repeated here merely for the sake of brevity.

The CNS disorder or disease or diabetes may be associated with an α3 subtype of the GABA_{A} receptor, such as the α3β2γ2 subtype of the GABA_{A} receptor. For example, said steroid-related CNS disorder or disease is selected from the group consisting of hyperphagia disorder; obesity; Prader-Willi's syndrome; polycystic ovarian syndrome; increased appetite disorder; obesity in diabetes; pathological food cravings; hypothalamic obesity; Cushing's syndrome; hyperphagia disorder associated with injury to the hypothalamus; alcoholims; substance use disorder; relapses into alcohol and/or substance use disorder; epilepsy; menstruation cycle dependent epilepsy; seizure disorder; worsening of Petit Mal epilepsy; memory disturbance; learning disturbance; menstrual cycle linked memory changes; stress related memory changes; stress related learning difficulties; hepatic encephalopathy; Down's syndrome; Alzheimer's disease; depression; stress related depression; premenstrual syndrome; premenstrual dysphoric disorder; menstrual cycle linked mood changes; negative mood such as as tension, irritability and depression; migraine; menstrual cycle linked migraine; stress linked migraine; hypersomnia and in particular stress related hypersomnia; sedation; idiopathic hypersomnia; sleep disorders; fatigue syndrome; burn-out syndrome; menstrual cycle linked sleep disorders; attention disorders; menstrual cycle linked difficulties in concentration; ADHD; mobility disorders; essential tremor; Tourette's syndrome; balance disturbances; disturbance of motor function; and clumsiness.

In particular, the disease or disorder may be selected from the group consisting of a steroid-related CNS disorder or disease and diabetes.

In particular, said disorder may be selected fro the group consisint of hyperphagia disorder; obesity; Prader-Willi's syndrome; and polycystic ovarian syndrome; increased appetite disorder; obesity in diabetes, pathological food cravings; hypothalamic obesity; Cushing's syndrome; hyperphagia disorder associated with injury to the hypothalamus; alcoholism, substance use disorder; relapses into alcohol and/or substance use disorder and diabetes, such as the group consisint of hyperphagia disorder; obesity; Prader-Willi's syndrome; and polycystic ovarian syndrome; increased appetite disorder; obesity in diabetes, pathological food cravings; hypothalamic obesity; Cushing's syndrome; hyperphagia disorder associated with injury to the hypothalamus; alcoholism, substance use disorder; relapses into alcohol and/or substance use disorder. In particular, the disease may be a disease selected from the group consisting of obesity, hyperphagia disorder, Prader-Willi's syndrome, polycystic ovarian syndrome, and diabetes. The disease may be Prader-Willi's syndrome. The disease may be polycystic ovarian syndrome, resulting in overweight or obesity. The disease may be diabetes, resulting in overweight or obesity. The disease may be obesity. The disease may be hyperphagia disorder. The disease may be alcoholims. The disease may be substance use disorder.

As explained above, 3α-ethynyl-3β-hydroxy-5α-androstan-17-methoxime and 3α-ethyl-3β-hydroxy-5α-androstan-17-oxime may be in the form of a compound or a pharmaceutically acceptable salt thereof. In one embodiment of the fifth aspect, said compound is in the form of a sodium salt. Other salts apparent to a person of skill in the art are also plausible as disclosed in connection with the first aspect.

Additionally there is provided a method of treating, alleviating and/or preventing a condition caused by exposure to at least one 3α-hydroxy-steroid, comprising administering a pharmaceutically effective amount of compound as disclosed herein, or a pharmaceutically acceptable salt, hydrate, prodrug or solvate thereof, to a patient in need thereof. Said exposure may be to an endogenous or exogenous 3α-hydroxy-steroid.

Additionally there is provided a method of treating, alleviating and/or preventing a side effect caused by an anti-inflammatory steroid, postmenopausal therapy, and/or an oral contraceptive, comprising administering a pharmaceutically effective amount of compound as defined herein, or a pharmaceutically acceptable salt, hydrate, prodrug or solvate thereof, to a patient in need thereof.

In one embodiment, the method of treatment, alleviation and/or prevention as disclosed herein results in a decrease of bodyweight. Plausibly, a decrease in bodyweight may be seen after 1 to 100 days, such as 2 to 20 days, such as after 3 to 15 days, such as after 5 to 10 days. In one embodiment, said method results in a decrease of daily calory intake by at least about 10%, such as at least 15%, such as at least 20%, such as at least 25%, such as at least 30%, such as at least 35%, such as at least 40%, such as at least 45%, such as at least 50%.

In one particular embodiment, there is provided a method as dislclosed herein, wherein said compound is administrated intravenously, nasally, per rectum, intravaginally, percutaneously, intramuscularly, or orally. In one embodiment, said administration is oral or nasal administration.

As discussed in detail in connection with the third aspect as disclosed herein, the compound as disclosed herein may be administrated in a dose of from about 0.1 to about 300 mg per kg body weight from about 0.2 to about 200 mg per kg body weight, such as a dose of from about 0.3 to about 150 mg, such as about 0.4 to about 150 mg per kg bodyweight, such as about 0.5 to about 120 mg per kg bodyweight, such as from about 1 to about 100 mg per kg body weight, such as from about 1 to about 50 mg per kg body weight, such from about 1 to about 5 mg per kg body weight, such as about 1 mg per kg body weight. In particular, said compound may be administrated at about 0.2 to about 200 mg per kg body weight.

As discussed in detail with the context of the third aspect and not repeated here for the sake of brevity, the compound of the present disclosure may provide an antagonistic effect on the effect of γ-aminobutyric acid (GABA) and/or any GABA_{A} receptor modulating steroids (GAMS) on the GABA_{A}-receptor α3 subtype(s). Thus in one embodiment, there is provided a method as disclosed herein, wherein said compound provides an antagonistic effect on the effect of γ-aminobutyric acid (GABA) and/or any GABA_{A} receptor modulating steroids (GAMS) on the GABA_{A}-receptor 03 subtype(s). In one embodiment, said antagonistic effect is on the effect of γ-aminobutyric acid (GABA) on the GABA_{A} receptor α3 subtype(s) or a on the effect of any GABA_{A} receptor modulating steroids (GAMS) on the GABA_{A}-receptor α3 subtype(s). Additionally, said compound may further provide an antagonistic effect on the effect of γ-aminobutyric acid (GABA) and/or any GABA_{A} receptor modulating steroids (GAMS) on the GABA_{A} receptor α1, α2, α4 and/or α5 subtype(s).

According to a sixth aspect of the invention, there is provided use of 3α-ethynyl-3β-hydroxy-5α-androstan-17-methoxime and/or 3α-ethyl-3β-hydroxy-5α-androstan-17-oxime or a pharmaceutically acceptable salt, hydrate, prodrug or solvate thereof, for the manufacture of a medicament for the prevention, alleviation and/or treatment steroid-related CNS disease or disorder, an autoimmune disease, and diabetes, for example a disease or disorder associated with an α3 subtype of the GABA_{A} receptor. In this aspect of the invention, the corresponding embodiments of the third aspect, are applicable and are not repeated here merely for the sake of brevity. In particular, the disease or disorder may be selected from the group consisting of a CNS disorder or disease, an autoimmune disease, or diabetes that is associated with an α3 subtype of the GABA_{A} receptor, such as the α3β2γ2 subtype of the GABA_{A} receptor.

In particular, said CNS disease or disorder may be selected from the group consisint of hyperphagia disorder; obesity; Prader-Willi's syndrome; and polycystic ovarian syndrome; increased appetite disorder; obesity in diabetes, pathological food cravings; hypothalamic obesity; Cushing's syndrome; hyperphagia disorder associated with injury to the hypothalamus; alcoholism, substance use disorder; relapses into alcohol and/or substance use disorder and diabetes, such as the group consisint of hyperphagia disorder; obesity; Prader-Willi's syndrome; and polycystic ovarian syndrome; increased appetite disorder; obesity in diabetes, pathological food cravings; hypothalamic obesity; Cushing's syndrome; hyperphagia disorder associated with injury to the hypothalamus; alcoholism, substance use disorder; relapses into alcohol and/or substance use disorder.

In particular, the disease or disorder may be a disease selected from the group consisting of obesity, hyperphagia disorder, Prader-Willi's syndrome, polycystic ovarian syndrome, and diabetes. The disease may be Prader-Willi's syndrome. The disease or disorder may be polycystic ovarian syndrome, resulting in overweight or obesity. The disease or disorder may be diabetes, resulting in overweight or obesity. The disease or disorder may be obesity. The disease or disorder may be hyperphagia disorder.

The compounds as disclosed herein may also be useful for cosmetic applications. Thus, in a seventh aspect of the present disclosure, there is provided a use a compound selected from the group consisting of 3α-ethynyl-3β-hydroxy-5α-androstan-17-methoxime according to Formula 1 and 3α-ethyl-3β-hydroxy-5α-androstan-17-oxime according to Formula 2, or a cosmetically acceptable salt, hydrate, precursor or solvate thereof, for non-medical reduction and/or prevention of overweight in a subject. Thus, said use in a non-therapeutic use and may also be referred to a cosmetic use. The skilled person appreciates that the terms non-medical, non-therapeutic and cosmetic are synonymous in this context and exclude medical uses which include treatment and/or preventions of pathological conditions. The compound can also be a precursor, which is transformed into 3α-ethynyl, 3β-hydroxy 5α-androstan-17-methoxime or into 3α-ethyl-3β-hydroxy-5α-androstan-17-oxime in the body of a subject, similarly to a prodrug. It is envisioned that prevention of overweight comprises reducing calory intake. For example, reduction of calory intake by at least about 10%, such as at least 15%, such as at least 20%, such as at least 25%, such as at least 30%, such as at least 35%, such as at least 40%, such as at least 45%, such as at least 50%.

To clarify, said non-medical use relates to reduction or prevention in subjects who have a BMI of less than 30. Thus, in one embodiment, said reduction and/or prevention of overweight is in a subject who has a BMI<30.

In one embodiment, said use relates to prevention of overweight in a subject who has a BMI below 25, and optionally who wishes to maintain a BMI in the range of from about 18.5 to 24.9.

In one embodiment, said use relates to reduction of overweight in a subject who has a BMI in the range of 25 to 29.9, and optionally who wishes to reduce the BMI to the range of from about 18.5 to 24.9.

The skilled person will appreciate that said compound may be administrated at a dose as disclosed in connection with the third aspect is equally applicable and is not repeated here for the sake of brevity. Similarly, the administration occasions disclosed in connection with the third aspect are also applicable here.

The present inventors envision a use of a composition comprising the compound as disclosed herein, wherein said composition is a cosmetic composition. Thus, there is provided a use of a cosmetic composition comprising 3α-ethynyl-3β-hydroxy-5α-androstan-17-methoxime and/or 3α-ethyl-3β-hydroxy-5α-androstan-17-oxime and at least one cosmetically acceptable excipient. The skilled person will appreciate the excipients disclosed in connection with the second aspect, relating to the pharmaceutical composition, also are applicable to the cosmetic composition and are not repeated here for the sake of brevity. Thus, said excipient are considered cosmetically acceptable excipients. Additionally, the skilled person is aware of other suitable cosmetic acceptable excipients.

The skilled person will appreciate that said compound may be administrated at a dose as disclosed in connection with the third aspect is equally applicable and is not repeated here for the sake of brevity. For clarity, thus said doses are considered cosmetically effective doses. However, in particular, it is envisioned that said 3α-ethynyl-3β-hydroxy-5α-androstan-17-methoxime and/or 3α-ethyl-3β-hydroxy-5α-androstan-17-oxime is administered by a route of administration selected from the group consisting of nasal, percutaneous, subcutaneous, transdermal, and oral administration

may be suitable administration routes. In one embodiment, said route of administration selected from the group consisting of nasal, transdermal, and oral administration. In one embodiment, said route of administration is oral administration.

It is envisioned that said use will lead to the decrease in bodyweight which may be seen after 1 to 100 days, such as 2 to 20 days, such as after 3 to 15 days, such as after 5 to 10 days. Thus, in one embodiment use results in a decrease in bodyweight after 1 to 100 days, such as 2 to 20 days, such as after 3 to 15 days, such as after 5 to 10 days, of treatment. In one particular embodiment, said use results in a decrease of daily calory intake by at least about 10%, such as at least 15%, such as at least 20%, such as at least 25%, such as at least 30%, such as at least 35%, such as at least 40%, such as at least 45%, such as at least 50%.

In a related aspect, there is provided a cosmetic, non-therapeutic method of preventing or reducing overweight in a subject comprising administering a cosmetically effective amount of a compound selected from the group consisting of 3α-ethynyl-3β-hydroxy-5α-androstan-17-methoxime according to Formula 1 and 3α-ethyl-3β-hydroxy-5α-androstan-17-oxime according to Formula 2, or a cosmetically acceptable salt, hydrate, precursor or solvate thereof. In one embodiment, there is provided said cosmetic, non-therapeutic method, wherein said prevention or reduction of overweight is in a subject having a BMI<30. In one embodiment, there is provided said cosmetic, non-therapeutic method, wherein said overweight is defined as a BMI in the range of 25-29.9.

In one embodiment of said cosmetic, non-therapeutic method, a decrease in bodyweight is seen after 1 to 20 days, such as after 3 to 15 days, such as after 5 to 10 days. The skilled person appreciates that embodiments disclosed in the context of the seventh aspect above are also applicable to the cosmetic, non-therapeutic method as disclosed herein.

In an eight aspect, there is provided a cosmetic composition comprising a cosmetically effective amount of a compound selected from the group consisting of 3α-ethynyl-3β-hydroxy-5α-androstan-17-methoxime as shown in Formula 1 and 3α-ethyl-3β-hydroxy-5α-androstan-17-oxime as shown in Formula 2 or a cosmetically acceptable salt, hydrate, precursor or solvate thereof and at least one cosmetically acceptable excipient. In one particular embodiment, said compound is 3α-ethynyl-3β-hydroxy-5α-androstan-17-methoxime or a cosmetically acceptable salt, hydrate, precursor or solvate thereof. In another embodiment, said compound is 3α-ethyl-3β-hydroxy-5α-androstan-17-oxime or a cosmetically acceptable salt, hydrate, precursor or solvate thereof.

It is envisioned that said composition may comprise both said compounds. Acceptable excipients are discussed in detail in the context of the third aspect and are not repeated there for the sake of brevity. The cosmetic compositon may be formulated for a route of administration selected from the group consisting of nasal, percutaneous, subcutaneous, transdermal, and oral administration, in particular group consisting of nasal, transdermal, and oral administration as dicussed above. In one embodiment, said cosmetic composition is formulated for oral or nasal administration.

It is noted that, as used in this specification and the appended claims, the singular forms “a”, “an”, and “the” also include plural referents unless the context clearly dictates otherwise.

The term “blocking” is meant to define an effect where in this case GABA or the 3α-hydroxy-5α/β-steroids are prevented from acting on the GABA-R receptor. It is to be understood that “blocking” is an entirely different effect than meant by “modulation” or “repression” or similar terms, which suggest that an action is still taking place, but to a lesser extent or at a slower rate.

The term “pharmaceutical composition” is used in its widest sense, encompassing all pharmaceutically applicable compositions containing at least one active substance and optional carriers, adjuvants, diluents, constituents etc.

The term “pharmaceutical composition” also encompasses a composition comprising the active substance in the form of derivate or a pro-drug, such as pharmaceutically acceptable salts, sulphates and esters. The manufacture of pharmaceutical compositions for different routes of administration falls within the capabilities of a person skilled in galenic chemistry.

The skilled person appreciates that a precursor is a compound that participates in a chemical reaction that produces another compound; and that a prodrug is a compound that, after intake, is metabolized (i.e. participates in a chemical reaction) within the body into a pharmacologically active drug (i.e. another compound). The terms “precursor” and/or “prodrug” are used herein to describe a compound that participates in a chemical reaction to form 3α-ethynyl-3β-hydroxy-5α-androstan-17-methoxime as shown in Formula 1 or 3α-ethyl-3β-hydroxy-5α-androstan-17-oxime as shown in Formula 2. Typically, the chemical reaction takes place after administration, or at administration of 3α-ethynyl-3β-hydroxy-5α-androstan-17-methoxime or 3α-ethyl-3β-hydroxy-5α-androstan-17-oxime. For example, a position of Formula 1 or 2 may be protected by a protection group. For example, the 3β-hydroxygroup of Formula 1 or 2 may be protected, thus forming a precursor or a prodrug. The skilled person appreciates that the prodrug or precursor may for example be activated intracellularly (for example via metabolic enzymes) and/or extracellularly (for example in the milieu of gastrointestinal fluids, within the systemic circulation and/or other extracellular fluid compartments or near therapeutic target tissues/cells, relying on common enzymes such as esterases and phosphatases or target directed enzymes).

The term “cosmetic composition” is used in its widest sense, encompassing all cosmetically applicable compositions containing at least one active substance and optional carriers, adjuvants, diluents, constituents etc. The term “cosmetic composition” also encompasses a composition comprising the active substance in the form of derivate or precursor form, such as cosmetically acceptable salts, sulphates and esters. The manufacture of cosmetical compositions for different routes of administration falls within the capabilities of a person skilled art.

The terms “administration” and “mode of administration” as well as “route of administration” are also used in their widest sense. The pharmaceutical composition and cosmetic composition of the present invention may be administered in a number of ways depending largely on whether a local, topical or systemic mode of administration is most appropriate for the condition be treated. These different modes of administration are for example topical (e.g., on the skin), local (including ophthalmic and to various mucous membranes, for example vaginal and rectal delivery), oral, parenteral or pulmonary, including the upper and lower airways. The preparation of such compositions and formulations is generally known to those skilled formulation arts and may be applied to the formulation of the composition of the present invention.

Exemplary compositions for oral administration include suspensions which can contain, for example, microcrystalline cellulose for imparting bulk, alginic acid or sodium alginate as a suspending agent, methylcellulose as a viscosity enhancer, and sweeteners or flavoring agents such as those known in the art, and immediate release tablets which can contain, for example, microcrystalline cellulose, dicalcium phosphate, starch, magnesium stearate, calcium sulfate, sorbitol, glucose and/or lactose and/or other excipients, binders, extenders, disintegrants, diluents and lubricants such as those known in the art. Suitable binders include starch, gelatin, natural sugars such as glucose or beta-lactose, corn sweeteners, natural and synthetic gums such as acacia, tragacanth or sodium alginate, carboxymethylcellulose, poly-ethylene glycol, waxes and the like. Disintegrators include without limitation starch, methylcellulose, agar, bentonite, xanthan gum and the like. The compound can also be delivered through the oral cavity by sublingual and/or buccal administration. Molded tablets, compressed tablets or freeze-dried tablets are exemplary forms which may be used. Exemplary compositions include those formulating the present compound(s) with fast dissolving diluents such as mannitol, lactose, sucrose and/or cyclodextrins. Also included in such formulations may be high molecular weight excipients such as celluloses (avicel) or polyethylene glycols (PEG).

Such formulations can also include an excipient to aid mucosal adhesion such as hydroxy propyl cellulose (HPC), hydroxy propyl methyl cellulose (HPMC), sodium carboxy methyl cellulose (SCMC), maleic anhydride copolymer (e.g., Gantrez), and agents to control release such as polyacrylic copolymer (e.g. Carbopol 934). Lubricants, glidants, flavors, coloring agents and stabilizers may also be added for ease of fabrication and use. Lubricants used in these dosage forms include sodium oleate, sodium stearate, magnesium stearate, sodium benzoate, sodium acetate, sodium chloride and the like. For oral administration in liquid form, the oral drug components can be combined with any oral, non-toxic, pharmaceutically or cosmetically acceptable inert carrier (where appropriate) such as ethanol, glycerol, water, and the like.

Compositions of the present invention suitable for oral administration may be presented as discrete units such as capsules, cachets, pills or tablets each containing a predetermined amount of the active ingredient; as a powder or granules; as a solution or a suspension in an aqueous liquid or a non-aqueous liquid, for example as elixirs, tinctures, suspensions or syrups; or as an oil-in-water liquid emulsion or a water-in-oil liquid emulsion. The active ingredient may also be presented as a bolus, electuary or paste.

A tablet may be made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, lubricating, surface active or dispersing agent. Molded tablets may be made by molding in a suitable machine a mixture of the powdered compound moistened with an inert liquid diluent. The tablets may optionally be coated or scored and may be formulated so as to provide slow or controlled release of the active ingredient therein. The present compounds can, for example, be administered in a form suitable for immediate release or extended release. Immediate release or extended release can be achieved by the use of suitable pharmaceutical or cosmetic compositions comprising the present compounds, or, particularly in the case of extended release, by the use of devices such as subcutaneous implants or osmotic pumps. The present compounds can also be administered liposomally.

Typical unit dosage compositions are those containing an effective dose, as hereinbefore recited, or an appropriate fraction thereof, of the active ingredient.

It should be understood that in addition to the ingredients particularly mentioned above, the compositions of this invention may include other agents conventional in the art having regard to the type of formulation in question, for example those suitable for oral administration may include flavoring agents.

With the term “antagonist” is meant a substance that hinders another substance, an agonist, to induce its effect. In this application the terms antagonist and blocker are used interchangably.

The term “obesity” refers to a condition in a patient having a BMI≥30. The BMI may be ≥35. The BMI may be ≥38. The BMI may be ≥40.

The term “overweight” refers to a condition in a subject having a BMI≥25 but <30.

The term “Prader-Willi's syndrome” refers to a condition in a patient having at least one error in chromosome 15. It also refers to a condition in a patient that is of similar symptomatology. This similar symptomatology forms the diagnosis of Prader-Willi syndrome in a subject that does not have a visible at least one error in chromosome 15. The skilled person is aware of the genetic mutations underlying Prader-Willi's syndrome and the symptomatology that forms the diagnosis of said syndrome in cases where the patient does not have a visible error on chromosome 15.

The term “Poly cystic ovarian syndrome” refers to a condition in a patient fulfilling the so called “Rotterdam criteria” as established by the American Society for Reproductive Medicine (ASRM) and the European Society for Human Reproduction and Embryology (ESHRE) at a meeting in Rotterdam in 2003.

The term “hyperphagia disorder” refers to an abnormally increased appetite for consumption of food. Hyperphagia disorder may be associated with injury to the hypothalamus.

The term “binge eating disorder” refers to a subject who suffers from recurrent episodes of eating large quantities of food and a feeling of loss of control. The full criteria for diagnoses of the disease is given in Diagnostic and Statistical Manual of Mental Disorders (DSM; latest edition: DSM-5, publ. 2013) as established by the American Psychiatric Association.

With the phrase “GAMSA” is meant compounds that only antagonize the action of positive GABA_{A} receptor modulating steroids. When such compounds only antagonize or block the action of positive GABA_{A} receptor modulating steroids, they have a “GAMSA effect”. The abbreviation “GAMSA” stands for GABA_{A} receptor modulating steroid antagonist.

As used herein, the terms 3α-ethynyl-3β-hydroxy-5α-androstan-17-methoxime and GR3053 are used interchangeably and means the same compound of Formula 1.

As used herein, the terms 3α-ethyl-3β-hydroxy-5α-androstan-17-oxime and GR3055 are used interchangeably and means the same compound of Formula 2.

As used herein, the term “patient” refers to an individual who is exhibits or is at risk of exhibiting symptom(s) of a disorder relating to obesity and/or hyperphagia disorder.

As used herein, the terms “α3 subtype GABA_{A} receptor” and “GABA_{A} receptor α3 subtype” are used interchangeably.

ff

As used herein, when the term “about” or “approximately” is used in relation to a numerical value, it is to be interpreted as a range of ±10%, such as ±9%, such as ±8%, such as 7%, such as ±6%, such as ±5%, such as 4%, such as ±3%, such as ±2%, such as ±1%. For example, when the value is stated to be about 10, this means that the value is in fact in the range of from 9 to 11, such as in the range of from 9.9 to 10.9, such as in the range of from 9.8 to 10.8, such as in the range of from 9.7 to 10.7, such as in the range of from 9.6 to 10.6, such as in the range of from 9.5 to 10.5, such as in the range of from 9.4 to 10.4, such as in the range of from 9.3 to 10.3, such as in the range of from 9.2 to 10.2, such as in the range of from 9.1 to 10.1.

The skilled person knows that numerical values relating to measurements are subject to measurement errors which place limits on their accuracy. For this reason, the general convention in the scientific and technical literature is applied: the last decimal place of a numerical value indicates its degree of accuracy. Where no other error margins are given, the maximum margin is ascertained by applying the rounding-off convention to the last decimal place e.g. for a measurement of 3.5 cm, the error margin is 3.45-3.54. When interpreting ranges of values in patent specifications, the skilled person proceeds on the same basis.

While the invention has been described with reference to various exemplary aspects and embodiments, it will be understood by those skilled in the art that various changes may be made and equivalents may be substituted for elements thereof without departing from the scope of the invention. Therefore, it is intended that the invention is not limited to any particular embodiment contemplated, but that the invention will include all embodiments falling within the scope of the appended claims. The invention will be further illustrated by the following non-limiting Examples.

### REFERENCES

Tian et al., 2019, J. Diabetes Res., doi: 5783545
Prud'homme et al., 2015, Autoimmun Rev., 11, 1048-56
Wang et al. 2000, Acta Physiol Scand., 169, 333-341
Wang et al. 2002, J Neurosci., 22(9):3366-75
Frye et al. 2000, Pharmacol Biochem Behav, 67, 587-596
Rose et al. 2018, Obesity. 26(11):1727-1732Bhandage et al. 2021, Cell Mol Life Sci, 78, 5667-5679
Lindquist et al. 2006, J Neurochem, 97(5):1349-1356
Olsen et al. 2009, Neuropharmacology, 56(1):141-148
Uusi-Oukari M et al, 2010, Pharmacol Rev, 62(1):97-135
Tian J et al. 2011, Autoimmunity, 44:465-470
Bhat R et al. 2010, Proc Natl Acad Sci USA, 107(6):2580-2585
Li J et al, 2017, Cell 168(1-2):86-100.e115

### BRIEF DESCRIPTION OF THE DRAWINGS

FIG. 1 shows the antagonistic effects by 3α-ethynyl-3β-hydroxy-5α-androstan-17-methoxime (GR3053) and 3α-ethyl-3β-hydroxy-5α-androstan-17-oxime (GR3055) on (A) THDOC enhanced GABA response and on (B) GABA mediated current response. Numbers of tested cells in the bars=n.

FIG. 2 shows the weight increase in grams in rats treated with 3α-ethynyl-3β-hydroxy-androstan-17-one for five days. Grey stables are the increase from arrival of the rat and white stables from the start of the treatment. It is shown that treated rats exhibited a lower increase in weight compared to the control group. FIG. 2A shows the increase from the start of treatment and FIG. 2B shows the increase from arrival.

FIG. 3 shows the difference in weight after 10 days of treatment of rats with two different doses of 3α-ethynyl-3β-hydroxy-5α-androstan-17-one, vehicle or estradiol+progesterone. The weight difference is normalized from the vehicle mean.

### EXAMPLE 1: SYNTHESIS OF STEROIDS OF THE INVENTION

### General Considerations

It has been identified that a reaction of the ethyl Grignard reagent with 3, 20/17 diketone steroids is in most cases selective for the position 3 and thus, no need for protection/deprotection for ketone functionality on carbon 20/17 is required.

Both 3α and 3β isomers are formed, which can be separated by chromatographic methods and recrystallized.

Starting materials for synthesizing 3α-ethyl-3β-hydroxy-5α-pregnan-20-one are the corresponding steroids with 3-hydroxy substituent and keto group in positions 20. They can be converted to the respective diones by oxidation with IBX reagent. The reaction proceeds smoothly and with complete conversion. Other suitable steroids can be employed as starting material when required such as 5α-androstane-3,17-dione. The reactions were carried out in suitable solvents such as methanol, ethanol, water, tetrahydrofuran (THF), diethyl ether, dichloromethane (DCM) or other solvents apparent to a person of skill in the art. When the reactants are chosen in a specific order it is possible to avoid, the use of toxic reactants, such as heavy metals, which are toxic even in traces or are difficult to be completely removed in the workup procedure.

Reactions involving air or moisture sensitive reagents, or products were carried out under inert atmosphere, such as nitrogen or argon gas, and in the presence of dry solvents. Diethyl ether and tetrahydrofuran were dried over Na in the presence of benzophenone. Syringes purged with inert gas were used for the transfer of reagents and dry solvents. Optimized time and temperature of the reactions were determined by monitoring the formation of products and the loss of starting material using a suitable chromatographic technique such as TLC or GC/MS.

Purifications were carried out by using chromatographic techniques such as flash silica chromatography or preparative high performance liquid chromatography (HPLC) by using a HPLC apparatus. Those skilled in the art can recognize that alternative purification methods can be employed, and laboratory chromatographic techniques can be adapted to industrial scale by using chromatographic columns for scaled preparations. Identification of the products are carried out by using suitable analytical techniques such as 1H-NMR, ¹³C-NMR, mass spectrometry, IR spectroscopy, X-ray spectroscopy and any other assay that one skilled in the art can recognize as suitable for structural identification and purity determination of 3α-ethyl-3β-hydroxy-5α-pregnan-20-one. A person skilled in the art will recognize that similar reagents, solvents, conditions, and parameters can be used in the reactions, depending on the substrate. NMR data are recorded using a Bruker 400 MHz spectrometer.

### Synthesis of: 3α-ethynyl-3β-hydroxy-5α-androstan-17-methoxime (Formula 1)

First Step: Synthesis of 3α-ethynyl-3,6-hydroxy-5α-androstan-17-one

3,17-5α-androstandione (5.0 mmol) was dissolved in 50 mL dry THF at room temperature (rt) under nitrogen. Ethynyl magnesium bromide (1.1 equiv) was added dropwise at rt under stirring and the solution was left stirring overnight at rt under nitrogen flow.

The yellowish solution was then quenched with saturated NH₄Cl_{(aq)} and the aqueous phase extracted with dichloromethane (3×30 mL). The collected organic phases were evaporated under reduced pressure. The resulting yellowish oil was dissolved in dichloromethane, washed with brine and dried over MgSO₄. The solution was reduced under vacuum, and the residue purified by silica flash column chromatography (1:4 diethylether:dichloromethane). Typical yields were 65%.

¹H NMR (400 MHz, CDCl₃): δ 2.40 (s, 1H); 2.37 (m, 1H); 2.0 (m, 1H); 0.79 (s, 3H); 0.77 (s, 3H).

Second Step: Synthesis of 3α-ethynyl-3,6-hydroxy-androstan-17-methoxime

A stirred mixture of 3α-ethynyl-3β-hydroxy-androstan-17-one (1.0 equiv.), MeONH₂·HCl (1.5 equiv.) and anhydrous NaOAc (1.5 equiv.) in absolute EtOH (2.5 mL) was treated with CeCl₃·7H2O (5 mol %). The reaction was heated to 50-80° C. without special protection against atmospheric oxygen and its progress was monitored by TLC. After consumption of the starting material, brine (10 mL) was added and the products were extracted with EtOAc (3×10 mL). The organic layers were combined, dried over MgSO₄ and concentrated under reduced pressure. The residue was purified by column chromatography, eluting with mixtures of hexanes and EtOAc. The experiment was conducted several times. Typical yields were 90%.

¹H NMR (400 MHz, CDCl₃-d₆): δ2.51-2.47 (m, 2H); 2.41 (s, 1H); 1.00 (m, 1H); 0.80 (m, 1H); 0.83 (s, 3H), 0.76 (s, 3H), 3.75 (s, 3H).

### Synthesis of 3α-ethyl-3β-hydroxy-5α-androstan-17-oxime (Formula 2)

First Step: Synthesis of 3α-ethynyl-3,6-hydroxy-5α-androstan-17-one

3,17-5α-androstandione (5.0 mmol) was dissolved in 50 mL dry THF at rt under nitrogen. Ethynyl magnesium bromide (1.1 equiv) was added dropwise at rt under stirring and the solution was left stirring overnight at rt under nitrogen flow.

The yellowish solution was then quenched with saturated NH₄Cl_{(aq)} and the aqueous phase extracted with dichloromethane (3×30 mL). The collected organic phases were evaporated under reduced pressure, the resulting yellowish oil dissolved in dichloromethane, washed with brine and dried over MgSO₄. The solution was reduced under vacuum, and the residue purified by silica flash column chromatography (1:4 diethylether:dichloromethane). The experiment was conducted several times. Typical yields were around 65%.

¹H NMR (400 MHz, CDCl₃): δ 2.40 (s, 1H); 2.37 (m, 1H); 2.0 (m, 1H); 0.79 (s, 3H); 0.77 (s, 3H).

Second Step: Synthesis of 3α-ethyl-3,6-hydroxy-5α-androstan-17-one

3α-ethynyl-3β-hydroxy-5α-androstan-17-one (98 mg, 0.31 mmol) was dissolved in 30 mL ethanol and 5 mL dichloromethane. Some drops glacial acetic acid, and a small amount Pd/C 10% were added to the solution.

Hydrogen (T=25° C. and P=1 Atm) was bubbled into the solution overnight while stirring. The resulting mixture was filtered over celite, yielding a clear solution, which was evaporated under reduced pressure. The resulting crude was dissolved in 10 mL dichloromethane and washed with an aqueous solution of NaHCO₃. The organic phases were then collected and dried over MgSO₄, filtered and the solvent removed under reduced pressure, yielding 90 mg (0.28 mmol, 91% yield) white crystals.

¹H NMR (400 MHz, CDCl₃): δ 2.37 (m, 1H); 2.0 (m, 1H); 0.81 (t, 3H); 0.79 (2× s, 6H).

Third Step: Synthesis of 3α-ethyl-3,6-hydroxy-5α-androstan-17-oxime (Formula 2)

3α-ethyl-3β-hydroxy-5α-androstan-17-one (10 mmol) was dissolved in dichloromethane 5 mL and ethanol 50 mL at and in air atmosphere, in a 250 mL round bottom flask. 4 equiv. of NH₂OH·HCl and 4 equiv. of sodium acetate were dissolved in 5 mL H₂O and were then added to the steroid solution. 20 mL of ethanol was added and the mixture was put on reflux overnight. The mixture was then cooled and the solvent removed under reduced pressure. The white residue is then treated with 50 mL H₂O and 50 mL dichloromethane, the aqueous phase extracted with 3×30 mL dichloromethane. The collected organic phases are then dried over MgSO₄, filtrated and the solvent removed under reduced pressure. The final residue was purified by silica flash column chromatography dichloromethane:diethyl ether 4:1. The experiment was conducted several times and typical yields were 95-100% (quantitative).

¹H NMR (400 MHz, CDCl₃): δ 2.37 (m, 1H); 2.0 (m, 1H); 1.00 (m, 1H); 0.86 (t, 3H); 0.83 (2× s, 6H).

### Example 2: TESTING OF GABA_{A} RECEPTOR EFFECTS OF 3α-ethynyl-3β-hydroxy-5α-androstan-17-methoxime and 3α-ethyl-3β-hydroxy-5α-androstan-17-oxime ON HUMAN α3β3γ2L GABA_{A} RECEPTOR SUBTYPE

Aim: To investigate the effect of 3α-ethynyl-3β-hydroxy-5α-androstan-17-methoxime and 3α-ethyl-3β-hydroxy-5α-androstan-17-oxime, respectively, on the GABA_{A} receptor function. Experiments were performed both in absence and in presence of the GAMS tetrahydrodeoxycorticosterone (THDOC), in the absence and presence of GABA. In these tests the protocol was optimized to be similar to the physiological conditions in the synaptic cleft.

### Materials and Methods

### Apparatus Used

Axonpatch 200B, Axon Instruments (Foster City, CA, USA)
BD Plastikpak 2 ml syringes, Becton Dickinsson S.A. (Madrid, Spain)
Borosilicate glass pipettes, OD 1.5 mm, ID 0.86 mm, 10 mm of length (Bergmanlabora AB, Danderyd, Sweden)
Digidata 1322A, Axon Instruments (Foster City, CA, USA)
Dynaflow Pro II Platform Zeiss Axiovert 25, Fluicell AB (Möndal, Sweden)
Dynaflow Resolve chip, Fluicell AB (Möndal, Sweden)
GraphPad Prism, GraphPad Software Inc. (San Diego, CA, USA)
Incubator Mini Galaxy A, Model number 500, LabRum Klimat AB (Stockholm, Sweden)
Patch CLAMP hardware; Axon Instruments (Foster City, CA, USA)
pCLAMP software; Axon Instruments (Foster City, CA, USA)
SPSS statistical package version PASW 22 (SPSS Inc. Chicago, IL, USA)

### Solutions and Chemicals

### EC-Solution (in mM):

137 NaCl, AnalaR, Normapur, Leuven, Belgien, Lot 10B230005
5.0 KCl, Scharlau, (Barcelona, Spain), Lot 71782
1.0 CaCl₂), Merck, (Darmstadt, Germany), Lot A609382526
1.2 MgCl2×6H2O, Scharlau, (Barcelona, Spain), Lot 61629
10 HEPES, Saveen Werner AB (Limhamn, Sweden), Lot 61915
10 glucose, VWR International, (Poole, England), Lot K33805414524
pH was adjusted with 1 M NaOH (Eka Chemicals AB, Bohus, Sweden) Lot 131004 to 7.4.
   IC-Solution with ATP (in mM):
3.0 NaCl, AnalaR, Normapur, Leuven, Belgien, Lot 10B230005
1.2 MgCl2×6H2O, Scharlau, (Barcelona, Spain), Lot A609382526
10 HEPES, Saveen Werner AB (Limhamn, Sweden), Lot 61915
1.0 EGTA, Sigma Chemical Co. (St. Louis, MO, USA)
140 Cs-gluconate, provided by Dr. Ragagnin, Umecrine AB, Umea, Sweden
2 Mg-ATP, Sigma Chemical Co. (St. Louis, MO, USA), Lot 010M51521V
pH was adjusted with 1 M CsOH Sigma Chemical Co. (St. Louis, MO, USA) to 7.2.
GABA (Gamma aminobutyric acid), Sigma Chemical Co. (St. Louis, MO, USA), Lot 081 K2064
THDOC, Sigma Chemical Co. (St. Louis, MO, USA), Lot 030M4041
Trypsin-EDTA 0.25% 1×, Invitrogen (Carlsbad, California, USA)
DMEM+Glutamax, Invitrogen (Carlsbad, California, USA)
KM (DMEM+GlutaMax, FBS (Foetal Bovine Serum), Penicillin-Streptomycin). All chemicals from Invitrogen (Carlsbad, California, USA).

Cell culture: Wild-type HEK-293 cells passage 4 (obtained from Pasteur Institute, Paris, France), permanently transfected with cDNA to express the human α3β3γ2L GABA_{A} receptor subtype, were seeded in cell bind culture flasks, grown and maintained at the temperature of +37° C., with a gas mixture of 5% CO₂ in the cell incubator. The transfected cells were used for patch-clamp experiments minimum one passages after defrosting and 3-5 days after seeding. The α3β3γ2L cells were seeded for maximal 15 times.

Electrophysiological recordings: Whole cell patch technique was used to record whole-cell currents from the cells. Patch electrodes were pulled from 1.5 mm O.D., 0.86 mm I.D. borosilicate capillary glass without filament. Typical electrodes had a resistance of 2-6 MO when filled with intracellular solutions. After compensation for liquid junction potential a steady holding potential of −17 mV was used in all experiments. The cells were added to the chip (see below) and kept in EC solution (see above). EC solution with or without steroids and GABA were applied by the Dynaflow™ system (see below). All experiments were performed at room temperature (21-23° C.).

Dynaflow™ system: Dynaflow™ system with Resolve chips was used for all patch-clamp experiments including steroids. The Resolve chip consists of a glass chip enclosed by a PEEK plastic top part and a supporting plastic bottom part. Both wells and channels are glass coated. The channel width is 150 μm and the height 50 μm. The well volume is 150 μL. Run time at the flow rate of 26 μL/min was 90 min. The pump settings were as follows: BD Plastikpak™ 2 mL syringe with inner diameter of 8.1 mm was used. The syringe pump flow rate for Resolve chip was 26 μL/min.

The Resolve chip allows synchronized control of switching between 16 experimental solutions. Laminar flow at each solution outlet of the microfluidic chip prevents mixing, and a computer-controlled stage motor is used to move the chip relative to the patch pipette, allowing for relatively rapid solution exchange around the membrane patch. PClamp 9.0 software, DigiData 1322A converter and AxonPatch 200B were used to generate command pulses and collect data. Clampfit 10.3, Prism 6.0 and excel were used for data analyzes.

GABA and steroids: GABA was dissolved in EC-solution by ultrasound for about 20 minutes at maximal 40° C. to the concentration of 10 mM.

THDOC was dissolved in ethanol 99.5% to the concentration of 0.2 mM and tested steroids dissolved in ethanol 99.5% to the concentration of 2 mM (with ultrasound). The final ethanol concentration was 0.1% in all end-solutions, including the wash solution (EC) and the solution with GABA alone. End-solutions are the solutions added into the wells of the chip.

Protocol: The binding site of THDOC is in the intracellular region of the receptor. To achieve stable response, the cell was exposed to steroid (THDOC and/or steroid) 20 seconds before GABA application. The standard protocol as follow: 20 s preincubation of steoids (100 nM THDOC alone or in combination with one of 1 μM 3α-ethynyl-3β-hydroxy-5α-androstan-17-methoxime and 3α-ethyl-3β-hydroxy-5α-androstan-17-oxime), followed by 2 s application of 100 μM GABA±steroids (100 nM THDOC alone or in combination with one of 1 μM 3α-ethynyl-3β-hydroxy-5α-androstan-17-methoxime and 3α-ethyl-3β-hydroxy-5α-androstan-17-oxime). Washout time was 120 s.

### Analysis

100 nM THDOC did not activate the α3β3γ2L subtype of the GABA_{A} receptor. Steroid (3α-ethynyl-3β-hydroxy-5α-androstan-17-methoxime or 3α-ethyl-3β-hydroxy-5α-androstan-17-oxime)+THDOC, in absence of GABA, were therefore not analyzed.

None of 3α-ethynyl-3β-hydroxy-5α-androstan-17-methoxime and 3α-ethyl-3β-hydroxy-5α-androstan-17-oxime activate the receptor in absence of GABA, therefore not analyzed.

In all experiments 100 μM GABA was used as the reference point for the GABA response. This value was subtracted from all resulting values with 3α-ethynyl-3β-hydroxy-5α-androstan-17-methoxime and 3α-ethyl-3β-hydroxy-5α-androstan-17-oxime. Each cell has its own reference point.
The effect of 1 μM 3α-ethynyl-3β-hydroxy-5α-androstan-17-methoxime or 3α-ethyl-3β-hydroxy-5α-androstan-17-oxime in presence of 100 nM+100 μM GABA was measured as area under curve. The effect of 100 μM GABA (area under curve) was subtracted from all values to receive the THDOC enhanced effect. This is the relative effect (%) to 100 nM THDOC enhancement (set as 0%).
The effect of 1 μM 3α-ethynyl-3β-hydroxy-5α-androstan-17-methoxime or 3α-ethyl-3β-hydroxy-5α-androstan-17-oxime in presence of 100 μM GABA was measured as area under curve, relative effect (%) to 100 μM GABA (set as 0%).

Statistical analysis: All tests with 1 μM of steroid (THDOC, 3α-ethynyl-3β-hydroxy-5α-androstan-17-methoxime and/or 3α-ethyl-3β-hydroxy-5α-androstan-17-oxime) were performed by the paired non-parametric Wilcoxon Signed Ranks Test (2 related samples).

All values in the text are displayed as mean±SEM. Significance is marked as follows: *=P≤0.05, **=P≤0.01, ***=P≤0.001, n=number of observations pooled form 10-20 cells. SPSS was used for statistical test of paired Wilcoxon Signed Ranks Test.

### Results

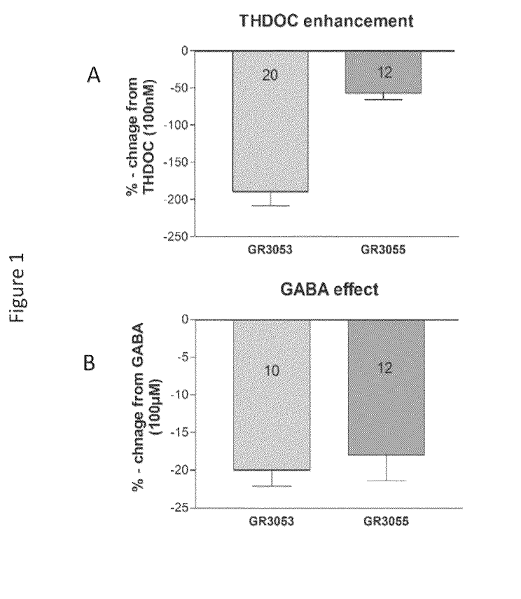

FIG. 1 shows the effects by 1 μM 3α-ethynyl-3β-hydroxy-5α-androstan-17-methoxime (GR3053) and 3α-ethyl-3β-hydroxy-5α-androstan-17-oxime (GR3055) on THDOC enhanced GABA response (100 nM=0%) and on GABA mediated current response. All tested steroids have an antagonistic effect on the 100 nM THDOC enhanced GABA mediated current response (see Table 2 and FIG. 1).

3α-ethynyl-3β-hydroxy-5α-androstan-17-methoxime completely block (>100%) the THDOC enhanced effect. Furthermore, 3α-ethynyl-3β-hydroxy-5α-androstan-17-methoxime had some antagonistic effect on GABAs own effect on the current. 3α-ethyl-3β-hydroxy-5α-androstan-17-oxime reduce the THDOC enhanced effect by−53%.

3α-ethynyl-3β-hydroxy-5α-androstan-17-methoxime and 3α-ethyl-3β-hydroxy-5α-androstan-17-oxime had a reducing effect of −20% and −18% respectively, on the effect provided by GABA. None of the tested steroids had an own effect, in absence of GABA and/or THDOC.

100 nM THDOC alone did not activate the α3β3γ2L GABA_{A} receptor in absence of GABA. Therefore, the baseline shift by THDOC alone was not measured.

TABLE 2
The effect by steroids (1 μM) on THDOC enhanced GABA response
and on GABA mediated current response.

| | Results: Mean ± SEM (P; N) | |
|---|---|---|
| | Area under curve | |
| | 100 nM THDOC | |
| Substance (1 μM) | enhancement | 100 μM GABA |
| 3α-ethynyl-3β-hydroxy-5α- | −190 ± 18.5%, | −20 ± 2.1% |
| androstan-17-methoxime | (P < 0.001; N = 20) | (P = 0.005; N = 10) |
| (GR3053) | | |
| 3α-ethyl-3β-hydroxy-5α- | −57 ± 9.0%, | −18 ± 3.4%, |
| androstan-17-oxime | (P = 0.003; N = 12) | (P = 0.006; N = 12) |
| (GR3055) | | |

Conclusion: Both tested substances have an antagonistic effect on THDOC enhanced GABA mediated current response.

3α-ethyl-3β-hydroxy-5α-androstan-17-oxime reduced the THDOC effect by 57% and reduced the GABA effect by 18%. The reducing effect on the GABA mediated current response shows that 3α-ethyl-3β-hydroxy-5α-androstan-17-oxime has a partial antagonistic effect on GABAs effect alone on current response. A reduction of the effect by up to 10% may be obtained by a placebo vehicle.

3α-ethynyl-3β-hydroxy-5α-androstan-17-methoxime reduced the THDOC effect by 190% and reduced the GABA effect by 20%. The reducing effect on the GABA mediated current response shows that 3α-ethynyl-3β-hydroxy-5α-androstan-17-methoxime has a partial antagonistic effect on GABAs effect alone on current response. A reduction of the effect by up to 10% may be obtained by a placebo vehicle.

### Example 3: EFFECT OF 3α-ethynyl-3β-hydroxy-5α-androstan-17-one ON WEIGHT INCREASE IN ANIMAL MODEL OF GROWING MALE RATS

### Treatment and Testing Schedule

Animals: Male Wistar rats were kept in group cages, with three animals per cage, from delivery and throughout the period of experiments. The individual animals were marked so that they could be identified throughout the experiment duration. The animals weight an average of 152 g upon arrival. A reversed light dark (12:12 h) cycle was used with the dark period onset at 0600 hrs. Altogether fifty-six (56) rats were used in this study (experiment 1+2). The animals were delivered from the breeder Taconic (Denmark). The study protocol was approved by the Regional Ethics Committee of Umea University, Sweden.

Feeding: Standard chow and water were available ad libitum. A reversed 12-h dark-light cycle with lights off at 10.00 am and lights on at 22.00 pm was used. For identification, the rats were marked with a permanent marker on the tail. To avoid the endogenous allopregnanolone fluctuations that are present in the estrous cycle of female rats, male rats were chosen (Frye et al. 2000). The animals were allowed to acclimatize for at least 3 weeks before start of the experimental sessions. During this period, the rats were repeatedly handled and allowed habituation to the new environment and to all new procedures, to minimize stress during the experiments.

### Experimental Design

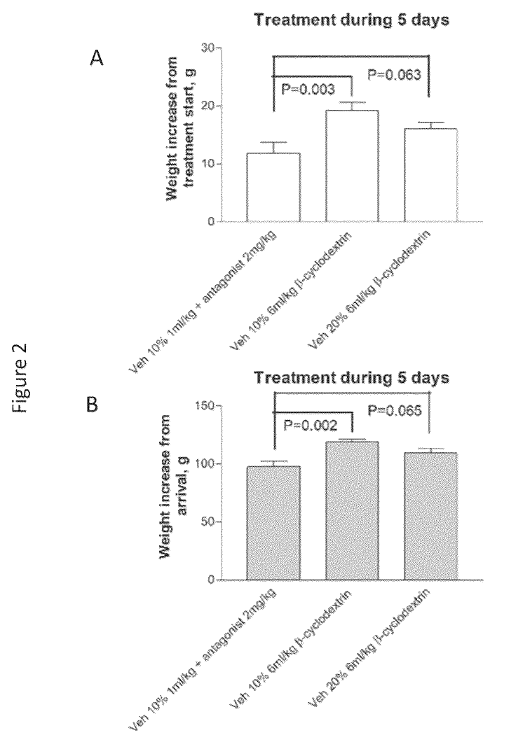

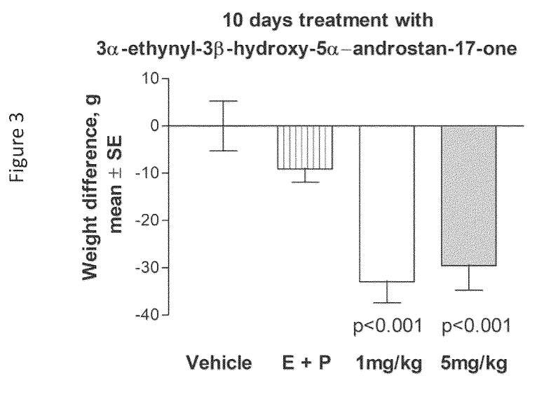

After 14 days of triad housing, handling and injection training of the animals, the animals were treated for 5 days with IV injections in experiment 1 and 10 days with a daily subcutaneous injection of the assigned treatment in experiment 2 (Table 3 and 4). The injections were given at 08.00 every morning. The animals were weighed the day before onset of the treatment and at the last day of injection i.e. on the 5th or 10th day of treatment. Weight was taken at the time of the last injection. A one-way ANOVA and non-parametric Kruscal-Wallice test showed differences for the treatment dosages compared to vehicle treatment. The results are shown in FIG. 2 (experiment 1) and FIG. 3 (experiment 2), respectively. Post hoc p-values are indicated in the Figures.

### Treatment Groups

TABLE 3
Treatment groups in experiment 1.

| | | |
|---|---|---|
| 3α-ethynyl-3β- | n = 7 | 3α-ethynyl-3β-hydroxy-5α- |
| hydroxy-5α- | | androstan-17-one, 2 mg/kg, |
| androstan-17- | | dissolved in 10% 2-Hydroxypropyl- |
| one | | β-cyclodextrin (Sigma-Aldrich), |
| | | to 2 mg/ml, given was 1 ml/kg. |
| Vehicle 1 | n = 7 | 10% 2-Hydroxypropyl-β- |
| | | cyclodextrin, 6 ml/kg |
| Vehicle 2 | n = 7 | 20% 2-Hydroxypropyl-β- |
| | | cyclodextrin, 6 ml/kg |

TABLE 4
Treatment groups in experiment 2.

| | | |
|---|---|---|
| Vehicle | n = 8 | s.c. vehicle (sesame oil, Sigma-Aldrich) |
| E + P Control | n = 9 | s.c. 5 mg/kg progesterone (P) + |
| | | 10 μg/kg 17β-estradiol (E) s.c. |
| | | Both Sigma-Aldrich |
| 3α-ethynyl- | n = 9 | s.c. 3α-ethynyl-3β-hydroxy-5α- |
| 3β-hydroxy- | | androstan-17-one; 1 mg/kg + 5 |
| 5α-androstan- | | mg/kg progesterone + 10 μg/kg |
| 17-one, low | | 17β-estradiol s.c. |
| 3α-ethynyl- | n = 9 | s.c. 3α-ethynyl-3β-hydroxy-5α- |
| 3β-hydroxy- | | androstan-17-one; 5 mg/kg + 5 |
| 5α-androstan- | | mg/kg progesterone + 10 μg/kg |
| 17-one, high | | 17β-estradiol |

### Results Example 2

Experiment 1: As can be seen in FIG. 2, there was no significant difference between the two vehicle treatments 10% or 20% 2-Hydroxypropyl-β-cyclodextrin. The increase in weight was significantly lower in the 3α-ethynyl-3β-hydroxy-5α-androstan-17-one-treated group than in the vehicle treated groups F(2,18)=6,293; p<0.008 when compared to weight at arrival to department or F(2,18)=6,103; p<0.009, or compared to weight at the start of treatment (Table 5). Ad hoc analysis of the weight increase from the arrival to the department shows a significant difference between 3α-ethynyl-3β-hydroxy-5α-androstan-17-one treatment and treatment with vehicle 10% (p<0.002) and a trend for significance against treatment with 20% β-cyclodextrin solution (p=0.065). The ad hoc test of the weight increases between start and end of treatment showed significant difference between the 3α-ethynyl-3β-hydroxy-5α-androstan-17-one treatment and vehicle treatment (p<0.003 for 10%) and a trend (p=0.063 for 20%). There was no significant difference between the weight changes in the vehicle treated groups. The cyclodextrin content in the 3α-ethynyl-3β-hydroxy-5α-androstan-17-one solution was 1 ml/kg while the cyclodextrin concentration in the vehicles was six times higher.

TABLE 5
Weight changes (mean, SEM gram) during
vehicle and 3α-ethynyl-3β-
hydroxy-5α-androstan-17-one treatment

| | | Mean weight | |
|---|---|---|---|
| | | increase since | |
| Treatment | N | arrival, g | SEM |
| 3α-ethynyl-3β-hydroxy-5α-androstan- | | | |
| 17-one, 2 mg/kg, 1 ml/kg, | 7 | 97.43 | 4.96 |
| β-cyclodextrine, 10% | | | |
| Vehicle 10% 6 ml/kg β-cyclodextrine | 7 | 118.57 | 2.78 |
| Vehicle 20% 6 ml/kg β-cyclodextrine | 7 | 109.14 | 4.60 |
| | | Mean weight | |
| | | increase since | |
| | | treatment start, | |
| | N | g. | SEM |
| 3α-ethynyl-3β-hydroxy-5α-androstan- | 7 | 11.86 | 1.83 |
| 17-one, 2 mg/kg, 1 ml/kg, | | | |
| β-cyclodextrine, 10% | | | |
| Vehicle 10% 6 ml/kg β-cyclodextrine | 7 | 19.14 | 1.39 |
| Vehicle 20% 6 ml/kg β-cyclodextrine | 7 | 16.00 | 1.13 |

FIG. 2 shows the mean±SEM weight changes in grams (g) the weight difference between the weight at the start of the treatment minus the weight at 5 five days later at the end of treatment (top, white stables) between the weight at arrival to the department minus the weight at the last day of treatment (bottom, grey stables). Antagonist 2 mg/kg=3α-ethynyl-3β3-hydroxy-5α-androstan-17-one 2 mg/kg.

Experiment 2: The results show of 3α-ethynyl-3β3-hydroxy-5α-androstan-17-one reduced the weight increase compared to controls treated with vehicle (placebo) or 5 mg/kg progesterone (P)+10 μg/kg 17β3-estradiol (E) sub cutaneous (s.c.). The reduction in weight was surprising large as the weight already after 10 days of treatment differed over 20% compared to vehicle, see FIG. 3. The results indicate that 3α-ethynyl-3β3-hydroxy-5α-androstan-17-one is very suitable to use as a treatment in e.g. adolescents with Prader-Willis syndrome.

Results and conclusion Example 3: FIG. 3 show the weight difference (Mean±SE) normalized from the vehicle and estradiol (E)+progesterone (P) control mean (set as 0±SE g weight) in the groups treated with two dosages of 3α-ethynyl-3β-hydroxy-5α-androstan-17-one. The results show that 3α-ethynyl-3β-hydroxy-5α-androstan-17-one reduced the weight increase compared to controls treated with vehicle (placebo). Both dose groups, 1 mg/kg (n=9) and 5 mg/kg (n=9) showed a significant reduction in weight compared to vehicle (−33±4.4 g; and −29,6±5.2 g resp. p<0.001 in both groups, FIG. 3). The 3α-ethynyl-3β-hydroxy-5α-androstan-17-one treated groups showed also a significantly reduced weight versus the E+P control rats (−23.9±4.4 g vs. −20.4±5.2 g p<0.001 and p=0.003, FIG. 3). The reduction in weight was surprisingly large as the weight already after 10 days of treatment differed over 20%. The results indicate that 3α-ethynyl-3β-hydroxy-5α-androstan-17-one is very suitable to use in prevention, alleviation or treatment of a disease associated with an α3 subtype of the GABA_{A} receptor, such as obesity, hyperphagia disorder, Prader-Willi's syndrome, polycystic ovarian syndrome, and/or diabetes. The present inventors consider that the compound of the invention to be particularly suitable for use in a patient with Prader-Willis syndrome, such as in adolescents with Prader-Willis syndrome.

### Example 4: EFFECT OF 3α-ethynyl-3β-hydroxy-5α-androstan-17-methoxime and 3α-ethyl-3β-hydroxy-5α-androstan-17-oxime ON WEIGHT INCREASE IN ANIMAL MODEL OF GROWING MALE RATS

In the same way as in example 3, the molecules of the present invention are investigated. 3α-ethynyl-3β-hydroxy-5α-androstan-17-one is replaced by 3α-ethynyl-3β-hydroxy-5α-androstan-17-methoxime and 3α-ethyl-3β-hydroxy-5α-androstan-17-oxime in an experiment with the same outline as example 3.

TABLE 6
Effect of 1 μM 3α-ethynyl-3β-hydroxy-5α-
androstan-17-one on receptor subtype
1β2γ2 and a3β3γ2. Experiment performed
essentially as described in Example 2.

| Steroids present | α1β2γ2 | α3β3γ2 | Result |
|---|---|---|---|
| 3α-ethynyl-3β- | −1.9 ± 2.4 % | −46 ± 1.3%, | Effect against GABAs |
| hydroxy-5α- | (NS; N = 22) | (P = 0.001; | agonistic effect |
| androstan-17-one | | N = 15) | |
| 1 μM | | | |
| 3α-ethynyl-3β- | No effect | −166 ± 19.9%, | Antagonism of 100 nM |
| hydroxy-5α- | | (P = 0.012; | THDOC-enhancement |
| androstan-17-one | | N = 8) | |
| 1 μM + THDOC | | | |

The molecules of the invention, 3α-ethynyl-3β-hydroxy-5α-androstan-17-methoxime and 3α-ethyl-3β-hydroxy-5α-androstan-17-oxime, are similar to the molecule of example 2, 3α-ethynyl-3β-hydroxy-5α-androstan-17-one. They all share the androstane core and, importantly, the 3α-ethyl-3β-hydroxyl stereochemistry. Furthermore, 3α-ethynyl-3β-hydroxy-5α-androstan-17-one has an antagonistic effect on γ-aminobutyric acid (GABA) and THDOC enhanced GABA signaling via the GABA_{A} receptor subypes 1β2γ2 and/or α3β3γ2 (see Table 6).

Without being bound by any theory, it is envisioned that the 3α-ethyl-3β-hydroxy stereochemistry is responsible for the effect on a α3 receptor subtype of the GABA_{A} receptor. Therefore, the skilled person will expect that 3α-ethynyl-3β-hydroxy-5α-androstan-17-methoxime and 3α-ethyl-3β-hydroxy-5α-androstan-17-oxime behaves similarly. 3α-ethynyl-3β-hydroxy-5α-androstan-17-methoxime and 3α-ethyl-3β-hydroxy-5α-androstan-17-oxime will thereby enable reduction in weight in a mammal.

The results will indicate that 3α-3α-ethynyl-3β-hydroxy-5α-androstan-17-methoxime and 3α-ethyl-3β-hydroxy-5α-androstan-17-oxime are very suitable to use in prevention, alleviation or treatment of a disease associated with an α3 subtype of the GABA_{A} receptor, such as obesity, hyperphagia disorder, Prader-Willi's syndrome, polycystic ovarian syndrome, and/or diabetes as well as in non-therapeutic treatment, prevention and/or alleviation of overweight. The present inventors consider that the compounds of the invention are to be particularly suitable for use in a patient with Prader-Willis syndrome, such as in adolescents with Prader-Willis syndrome.

### ITEMIZED LIST OF EMBODIMENTS

1. A compound selected from the group consisting of
3α-ethynyl-3β-hydroxy-5α-androstan-17-methoxime as shown in Formula 1

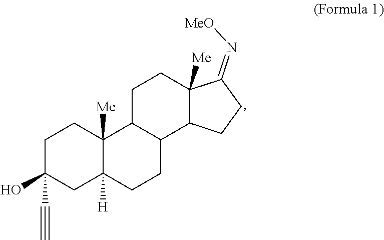 [SMILES: C#C[C@]1(O)CC[C@]2(C)C3CC[C@]4(C)/C(=N\OC)CCC4C3CC[C@H]2C1]

3α-ethyl-3β-hydroxy-5α-androstan-17-oxime as shown in Formula 2

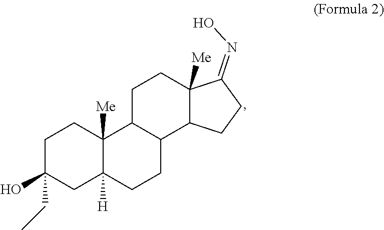 [SMILES: CC[C@]1(O)CC[C@]2(C)C3CC[C@]4(C)/C(=N\O)CCC4C3CC[C@H]2C1]

or
a pharmaceutically acceptable salt, hydrate, prodrug or solvate thereof or a cosmetically acceptable salt, hydrate, precursor or solvate thereof.

2. The compound according to item 1, wherein said compound is 3α-ethynyl-3β-hydroxy-5α-androstan-17-methoxime or a pharmaceutically acceptable salt, hydrate, prodrug or solvate thereof or a cosmetically acceptable salt, hydrate, precursor or solvate thereof.

3. The compound according to item 1, wherein said compound is 3α-ethyl-3β-hydroxy-5α-androstan-17-oxime, or a pharmaceutically acceptable salt, hydrate, prodrug or solvate thereof or a cosmetically acceptable salt, hydrate, precursor or solvate thereof.

4. The compound according to any one of items 1 to 3, wherein said compound or a pharmaceutically acceptable salt, hydrate, prodrug or solvate thereof or a cosmetically acceptable salt, hydrate, precursor or solvate thereof comprises ³H isotopes of hydrogen.

5. The compound according to any one of items 1 to 3, wherein said compound or a pharmaceutically acceptable salt, hydrate, prodrug or solvate thereof or a cosmetically acceptable salt, hydrate, precursor or solvate thereof comprises ²H isotopes of hydrogen.

6. The compound according to any one of items 1 to 5, wherein said pharmaceutically or cosmetically acceptable salt is a sodium salt.

7. A compound according to any one of items 1 to 6, for use as a medicament.

8. A compound according to any one of items 1 to 6, for use in prevention, alleviation and/or treatment of a steroid-related CNS disorder or disease, of an autoimmune disease, and/or of diabetes.

9. The compound for use according to item 8, wherein said steroid-related CNS disorder or disease is selected from the group consisting of hyperphagia disorder; obesity; Prader-Willi's syndrome; polycystic ovarian syndrome; increased appetite disorder; obesity in diabetes; pathological food cravings; hypothalamic obesity; Cushing's syndrome; hyperphagia disorder associated with injury to the hypothalamus; alcoholism; substance use disorder; relapses into alcohol and/or substance use disorder; epilepsy; menstruation cycle dependent epilepsy; seizure disorder; worsening of Petit Mal epilepsy; memory disturbance; learning disturbance; menstrual cycle linked memory changes; stress related memory changes; stress related learning difficulties; hepatic encephalopathy; Down's syndrome; Alzheimer's disease; depression; stress related depression; premenstrual syndrome; premenstrual dysphoric disorder; menstrual cycle linked mood changes; negative mood such as as tension, irritability and depression; migraine; menstrual cycle linked migraine; stress linked migraine; hypersomnia and in particular stress related hypersomnia; sedation; idiopathic hypersomnia; sleep disorders; fatigue syndrome; burn-out syndrome; menstrual cycle linked sleep disorders; attention disorders; menstrual cycle linked difficulties in concentration; ADHD; mobility disorders; essential tremor; Tourette's syndrome; balance disturbances; disturbance of motor function; and clumsiness.

10. The compound for use according to item 8 or 9, wherein said CNS disorder or disease, autoimmune disease, and/or diabetes is associated with an α3 subtype of the GABA_{A} receptor, such as the α3β2γ2 subtype of the GABA_{A} receptor.

11. The compound for use according to any one of items 8 to 10, wherein said steroid-related CNS disorder or disease is selected from the group consisting of hyperphagia disorder; obesity; Prader-Willi's syndrome; polycystic ovarian syndrome; increased appetite disorder; obesity in diabetes; pathological food cravings; hypothalamic obesity; Cushing's syndrome; hyperphagia disorder associated with injury to the hypothalamus; alcoholims; substance use disorder; relapses into alcohol and/or substance use disorder, such as group consisting of hyperphagia disorder; obesity; Prader-Willi's syndrome; polycystic ovarian syndrome; increased appetite disorder; obesity in diabetes; pathological food cravings; hypothalamic obesity; Cushing's syndrome and hyperphagia disorder associated with injury to the hypothalamus.

12. The compound for use according to any one of items 8 to 11, wherein said steroid-related CNS disorder or disease is obesity.

13. The compound for use according to any one of items 8 to 11, wherein said steroid-related CNS disorder is hyperphagia disorder.

14. The compound for use according to any one of items 8 to 11, wherein said steroid-related CNS disorder or disease is Prader-Willi's syndrome.

15. The compound for use according to any one of items 8 to 11, wherein said steroid-related CNS disorder or disease is polycystic ovarian syndrome.

16. The compound for use according to any one of items 8, 10 and 11, wherein said diabetes is diabetes type II.

17. The compound for use according to any one of items 8 to 11, wherein said steroid-related CNS disorder or disease is alcoholism or substance use disorder.

18. A compound according to any one of items 1 to 6, for use in prevention, alleviation and/or treatment of a condition caused by exposure to at least one endogenous or exogenous 3α-hydroxy-steroid.

19. A compound according to any one of items 1 to 6, for use in prevention, alleviation and/or treatment of a side effect caused by an anti-inflammatory steroid, postmenopausal therapy, and/or an oral contraceptive.

20. The compound for use according to any one according to items 11 to 16, wherein said use results in a decrease in bodyweight, optionally wherein said decrease is seen after 1 to 20 days, such as after 3 to 15 days, such as after 5 to 10 days.

21. The compound for use according to any one of items 7 to 20, wherein said compound is administrated intravenously, nasally, per rectum, intravaginally, percutaneously, intramuscularly, or orally.

22. The compound for use according to item 21, wherein said administration is oral or nasal administration.

23. The compound for use according to any one of items 7 to 22, wherein said compound is administrated in a dose of from about 0.1 to about 300 mg per kg body weight, such as from about 0.2 to about 200 mg per kg body weight, such as a dose of from about 0.3 to about 150 mg, such as about 0.4 to about 150 mg per kg bodyweight, such as about 0.5 to about 120 mg per kg bodyweight, such as from about 1 to about 100 mg per kg body weight, such as from about 1 to about 50 mg per kg body weight, such from about 1 to about 5 mg per kg body weight, such as about 1 mg per kg body weight.

24. The compound for use according to item 23, wherein said dose is from about 0.2 to about 200 mg per kg body weight.

25. The compound according to any one of items 1 to 6 or the compound for use according to any one of items 7 to 24, wherein said compound provides an antagonistic effect on the effect of γ-aminobutyric acid (GABA) and/or any GABA_{A} receptor modulating steroids (GAMS) on the GABA_{A}-receptor α3 subtype(s).

26. The compound according to any one of items 1 to 6 and 25 or the compound for use according to any one of items 7 to 25, wherein said compound provides an antagonistic effect on the effect of γ-aminobutyric acid (GABA) on the GABA_{A} receptor α3 subtype(s).

27. The compound according to any one of items 1 to 6 and 25 or the compound for use according to any one of items 7 to 26, wherein said compound provides an antagonistic effect on the effect of any GABA_{A} receptor modulating steroids (GAMS) on the GABA_{A}-receptor α3 subtype(s).

28. The compound according to any one of items 1 to 6 and 25 to 27 or the compound for use according to any one of items 7 to 27, wherein said compound further provides an antagonistic effect on the effect of γ-aminobutyric acid (GABA) and/or any GABA_{A} receptor modulating steroids (GAMS) on the GABA_{A} receptor α1, α2, α4 and/or α5 subtype(s).

29. A pharmaceutical composition comprising a therapeutically effective amount of a compound as according to any one of items 1 to 6 or a compound for use according to any one of items 7 to 28, or a pharmaceutically acceptable salt, hydrate, prodrug or solvate thereof, and at least one pharmaceutically acceptable excipient.

30. A method of treating, alleviating and/or preventing a steroid-related CNS disorder, an autoimmune disease, and/or diabetes, comprising administering a pharmaceutically effective amount of compound selected from the group consisting of
3α-ethynyl-3β-hydroxy-5α-androstan-17-methoxime as shown in Formula 1

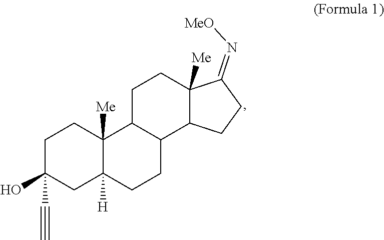 [SMILES: C#C[C@]1(O)CC[C@]2(C)C3CC[C@]4(C)/C(=N\OC)CCC4C3CC[C@H]2C1]

and
3α-ethyl-3β-hydroxy-5α-androstan-17-oxime as shown in Formula 2

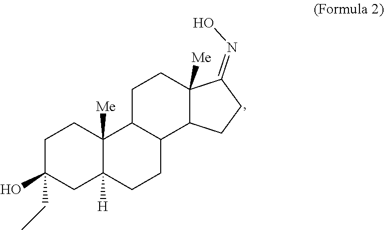 [SMILES: CC[C@]1(O)CC[C@]2(C)C3CC[C@]4(C)/C(=N\O)CCC4C3CC[C@H]2C1]

a pharmaceutically acceptable salt, hydrate, prodrug or solvate thereof, to a patient in need thereof.

31. The method of treating, alleviating and/or preventing according to item 30, wherein said steroid-related CNS disorder or disease is selected from the group consisting of hyperphagia disorder; obesity; Prader-Willi's syndrome; polycystic ovarian syndrome; increased appetite disorder; obesity in diabetes; pathological food cravings; hypothalamic obesity; Cushing's syndrome; hyperphagia disorder associated with injury to the hypothalamus; alcoholims; substance use disorder; relapses into alcohol and/or substance use disorder; epilepsy; menstruation cycle dependent epilepsy; seizure disorder; worsening of Petit Mal epilepsy; memory disturbance; learning disturbance; menstrual cycle linked memory changes; stress related memory changes; stress related learning difficulties; hepatic encephalopathy; Down's syndrome; Alzheimer's disease; depression; stress related depression; premenstrual syndrome; premenstrual dysphoric disorder; menstrual cycle linked mood changes; negative mood such as as tension, irritability and depression; migraine; menstrual cycle linked migraine; stress linked migraine; hypersomnia and in particular stress related hypersomnia; sedation; idiopathic hypersomnia; sleep disorders; fatigue syndrome; burn-out syndrome; menstrual cycle linked sleep disorders; attention disorders; menstrual cycle linked difficulties in concentration; ADHD; mobility disorders; essential tremor; Tourette's syndrome; balance disturbances; disturbance of motor function; and clumsiness.

32. The method of treating, alleviating and/or preventing according to any one of items 30 to 31, wherein said CNS disorder, autoimmune disease and/or diabetes is associated with an α3 subtype of the GABA_{A} receptor, such as the α3β2γ2 subtype of the GABA_{A} receptor.

33. The method of treating, alleviating and/or preventing according to any one of item 30 to 32, wherein said steroid-related CNS disorder or disease is selected from the group consisting of hyperphagia disorder; obesity; Prader-Willi's syndrome; polycystic ovarian syndrome; increased appetite disorder; obesity in diabetes; pathological food cravings; hypothalamic obesity; Cushing's syndrome; hyperphagia disorder associated with injury to the hypothalamus; alcoholims; substance use disorder; relapses into alcohol and/or substance use disorder, such as group consisting of hyperphagia disorder; obesity; Prader-Willi's syndrome; polycystic ovarian syndrome; increased appetite disorder; obesity in diabetes; pathological food cravings; hypothalamic obesity; Cushing's syndrome and hyperphagia disorder associated with injury to the hypothalamus.

34. The method of treating, alleviating and/or preventing according to any one of items 30 to 33, wherein said steroid-related CNS disorder or disease is obesity.

35. The method of treating, alleviating and/or preventing according to any one of items 30 to 33, wherein said steroid-related CNS disorder is a hyperphagia disorder.

36. The method of treating, alleviating and/or preventing according to any one of items 30 to 33, wherein said steroid-related CNS disorder or disease is Prader-Willi's syndrome.

37. The method of treating, alleviating and/or preventing according to any one of items 30 to 33, wherein said steroid-related CNS disorder or disease is polycystic ovarian syndrome.

38. The method of treating, alleviating and/or preventing according to any one of items 30 and 32, wherein said diabetes is diabetes type II.

39. The method of treating, alleviating and/or preventing according to any one of items 30 to 33, said steroid-related CNS disorder or disease is alcoholism or substance use disorder.

40. A method of treating, alleviating and/or preventing a condition caused by exposure to at least one 3α-hydroxy-steroid, comprising administering a pharmaceutically effective amount of compound according to any one of claims 1 to 6, or a pharmaceutically acceptable salt, hydrate, prodrug or solvate thereof to a patient in need thereof.

41. A method of treating, alleviating and/or preventing a side effect caused by an anti-inflammatory steroid, postmenopausal therapy, and/or an oral contraceptive, comprising administering a pharmaceutically effective amount of compound according to any one of claims 1 to 6, or a pharmaceutically acceptable salt, hydrate, prodrug or solvate thereof, to a patient in need thereof.

42. The method of treating, alleviating and/or preventing according to any one according to items 30 to 41, wherein said method results in a decrease in bodyweight, optionally wherein said descrese is seen after 1 to 20 days, such as after 3 to 15 days, such as after 5 to 10 days.

43. The method of treating, alleviating and/or preventing according to any one of items 30 to 42, wherein said compound is administrated intravenously, nasally, per rectum, intravaginally, percutaneously, intramuscularly, or orally.

44. The method of treating, alleviating and/or preventing according to item 43, wherein said administration is oral or nasal administration.

45. The method of treating, alleviating and/or preventing according to any one of items 30 to 44, wherein said compound is administrated in a dose of from about 0.1 to about 300 mg per kg body weight from about 0.2 to about 200 mg per kg body weight, such as a dose of from about 0.3 to about 150 mg, such as about 0.4 to about 150 mg per kg bodyweight, such as about 0.5 to about 120 mg per kg bodyweight, such as from about 1 to about 100 mg per kg body weight, such as from about 1 to about 50 mg per kg body weight, such from about 1 to about 5 mg per kg body weight, such as about 1 mg per kg body weight.

46. The method of treating, alleviating and/or preventing according to any one of items 31 to 45, wherein said dose from about 0.2 to about 200 mg per kg body weight.

47. The method of treating, alleviating and/or preventing according to any one of items 30 to 46, wherein said compound provides an antagonistic effect on the effect of γ-aminobutyric acid (GABA) and/or any GABA_{A} receptor modulating steroids (GAMS) on the GABA_{A}-receptor α3 subtype(s).

48. The method of treating, alleviating and/or preventing according to any one of items 30 to 47, wherein said compound wherein said compound provides an antagonistic effect on the effect of γ-aminobutyric acid (GABA) on the GABA_{A} receptor α3 subtype(s).

49. The method of treating, alleviating and/or preventing according to any one of items 30 to 47, wherein said compound provides an antagonistic effect on the effect of any GABA_{A} receptor modulating steroids (GAMS) on the GABA_{A}-receptor α3 subtype(s).

50. The method of treating, alleviating and/or preventing according to any one of items 30 to 49, wherein said compound further provides an antagonistic effect on the effect of γ-aminobutyric acid (GABA) and/or any GABA_{A} receptor modulating steroids (GAMS) on the GABA_{A} receptor α1, α2, α4 and/or α5 subtype(s).

51. Use of a compound according to any one of items 1 to 6, or a pharmaceutically acceptable salt, hydrate, prodrug or solvate thereof in the preparation of a medicament for treating, alleviating and/or preventing a of a steroid-related CNS disorder or disease, an autoimmune disease, and/or diabetes.

52. Use of a compound according to item 51, wherein said steroid-related CNS disorder is selected from the group consisting of hyperphagia disorder; obesity; Prader-Willi's syndrome; polycystic ovarian syndrome; increased appetite disorder; obesity in diabetes; pathological food cravings; hypothalamic obesity; Cushing's syndrome; hyperphagia disorder associated with injury to the hypothalamus; alcoholims; substance use disorder; relapses into alcohol and/or substance use disorder; epilepsy; menstruation cycle dependent epilepsy; seizure disorder; worsening of Petit Mal epilepsy; memory disturbance; learning disturbance; menstrual cycle linked memory changes; stress related memory changes; stress related learning difficulties; hepatic encephalopathy; Down's syndrome; Alzheimer's disease; depression; stress related depression; premenstrual syndrome; premenstrual dysphoric disorder; menstrual cycle linked mood changes; negative mood such as as tension, irritability and depression; migraine; menstrual cycle linked migraine; stress linked migraine; hypersomnia and in particular stress related hypersomnia; sedation; idiopathic hypersomnia; sleep disorders; fatigue syndrome; burn-out syndrome; menstrual cycle linked sleep disorders; attention disorders; menstrual cycle linked difficulties in concentration; ADHD; mobility disorders; essential tremor; Tourette's syndrome; balance disturbances; disturbance of motor function; and clumsiness.

53. Use of a compound according to any one of items 50 to 51, wherein said CNS disorder or disease, autoimmune disease and/or diabetes is associated with an α3 subtype of the GABA_{A} receptor, such as the α3β2γ2 subtype of the GABA_{A} receptor.

54. Use of a compound according to any one of items 50 to 53, wherein said steroid-related CNS disorder or disease is selected from the group consisting of hyperphagia disorder; obesity; Prader-Willi's syndrome; and polycystic ovarian syndrome; increased appetite disorder; obesity in diabetes, pathological food cravings; hypothalamic obesity; Cushing's syndrome; hyperphagia disorder associated with injury to the hypothalamus; alcoholism, substance use disorder; relapses into alcohol and/or substance use disorder.

55. Use of a compound according to any one of items 1-6 or a cosmetically acceptable salt, hydrate, precursor or solvate thereof for non-therapeutic prevention and/or reduction of overweight.

56. Use of compound according to item 55, wherein said prevention or reduction of overweight is in a subject having a BMI<30.

57. Use of compound according to item 55 or 56, wherein said overweight is defined as a BMI in the range of 25-29.9.

58. Method of preventing or reducing overweight in a subject comprising administering a cosmetically effective amount of compound according to any one of items 1 to 6 or a cosmetically acceptable salt, hydrate, precursor or solvate thereof.

59. Method of preventing or reducing overweight according to item 58, wherein said prevention or reduction of overweight is in a subject having a BMI<30.

60. Method of preventing or reducing overweight according to item 58 or 59, wherein said overweight is defined as a BMI in the range of 25-29.9.

61. The use according to any one of items 55 to 57 or the method of preventing or reducing overweight according to any one according to items 58 to 60, wherein a decrease in bodyweight is seen after 1 to 20 days, such as after 3 to 15 days, such as after 5 to 10 days.

62. A cosmetic composition comprising a cosmetically effective amount of a compound selected from the group consisting of
3α-ethynyl-3β-hydroxy-5α-androstan-17-methoxime as shown in Formula 1

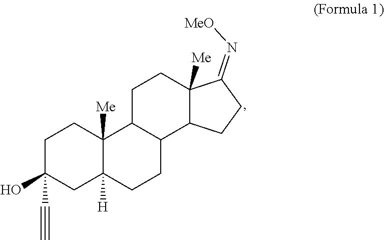 [SMILES: C#C[C@]1(O)CC[C@]2(C)C3CC[C@]4(C)/C(=N\OC)CCC4C3CC[C@H]2C1]

and
3α-ethyl-33-hydroxy-Sa-androstan-17-oxime as shown in Formula 2

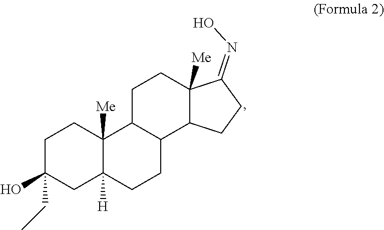 [SMILES: CC[C@]1(O)CC[C@]2(C)C3CC[C@]4(C)/C(=N\O)CCC4C3CC[C@H]2C1]

or a cosmetically acceptable salt, hydrate, precursor or solvate thereof and
at least one cosmetically acceptable excipient.

1. A compound, 3α-ethynyl-3β-hydroxy-5α-androstan-17-methoxime, as shown in Formula 1 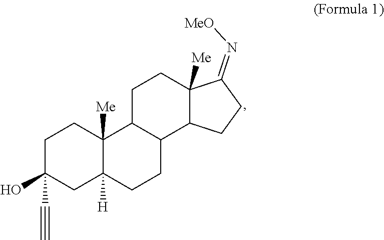 [SMILES: C#C[C@]1(O)CC[C@]2(C)C3CC[C@]4(C)/C(=N\OC)CCC4C3CC[C@H]2C1]
or
a pharmaceutically acceptable salt, hydrate, prodrug or solvate thereof or a cosmetically acceptable salt, hydrate, precursor or solvate thereof.

2. (canceled)

3. The compound according to claim 1, wherein the compound or a pharmaceutically acceptable salt, hydrate, prodrug or solvate thereof or a cosmetically acceptable salt, hydrate, precursor or solvate thereof comprises ³H isotopes of hydrogen or ²H isotopes of hydrogen.

4. (canceled)

5. The compound according to claim 1, wherein the pharmaceutically or cosmetically acceptable salt is a sodium salt.

6-27. (canceled)

28. A pharmaceutical or cosmetic composition comprising a therapeutically or cosmetically effective amount of a compound as defined in claim 1, or a pharmaceutically or cosmetically acceptable salt, hydrate, prodrug precursor or solvate thereof, and at least one pharmaceutically acceptable excipient or at least one cosmetically acceptable excipient.

29. (canceled)

30. A method of treating, alleviating and/or preventing a steroid-related CNS disorder, an autoimmune disease, and/or diabetes: or a condition caused by exposure to at least one 3α-hydroxy-steroid; or a side effect caused by an anti-inflammatory steroid, postmenopausal therapy, and/or an oral contraceptive, comprising administering a pharmaceutically effective amount of 3α-ethynyl-3β-hydroxy-5α-androstan-17-methoxime as shown in Formula 1 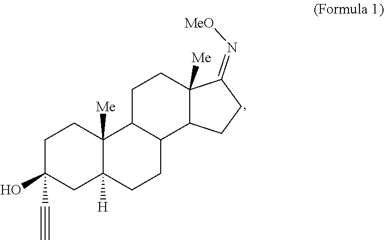 [SMILES: C#C[C@]1(O)CC[C@]2(C)C3CC[C@]4(C)/C(=N\OC)CCC4C3CC[C@H]2C1]
or a pharmaceutically acceptable salt, hydrate, prodrug or solvate thereof, to a patient in need thereof.

31. The method of treating, alleviating and/or preventing according to claim 30, wherein the steroid-related CNS disorder or disease is selected from the group consisting of hyperphagia disorder; obesity; Prader-Willi's syndrome; polycystic ovarian syndrome; increased appetite disorder; obesity in diabetes; pathological food cravings; hypothalamic obesity; Cushing's syndrome; hyperphagia disorder associated with injury to the hypothalamus; alcoholism; substance use disorder; relapses into alcohol and/or substance use disorder; epilepsy; menstruation cycle dependent epilepsy; seizure disorder; worsening of Petit Mal epilepsy; memory disturbance; learning disturbance; menstrual cycle linked memory changes; stress related memory changes; stress related learning difficulties; hepatic encephalopathy; Down's syndrome; Alzheimer's disease; depression; stress related depression; premenstrual syndrome; premenstrual dysphoric disorder; menstrual cycle linked mood changes; negative mood such as tension, irritability and depression; migraine; menstrual cycle linked migraine; stress linked migraine; hypersomnia and in particular stress related hypersomnia; sedation; idiopathic hypersomnia; sleep disorders; fatigue syndrome; burn-out syndrome; menstrual cycle linked sleep disorders; attention disorders; menstrual cycle linked difficulties in concentration; ADHD; mobility disorders; essential tremor; Tourette's syndrome; balance disturbances; disturbance of motor function; and clumsiness.

32. The method of treating, alleviating and/or preventing according to claim 30, wherein the CNS disorder, autoimmune disease and/or diabetes is associated with an α3 subtype of the GABA_{A} receptor, such as the α3β2γ2 subtype of the GABA_{A} receptor.

33. The method of treating, alleviating and/or preventing according to claim 30, wherein the steroid-related CNS disorder or disease is selected from the group consisting of hyperphagia disorder; obesity; Prader-Willi's syndrome; polycystic ovarian syndrome; increased appetite disorder; obesity in diabetes; pathological food cravings; hypothalamic obesity; Cushing's syndrome; hyperphagia disorder associated with injury to the hypothalamus; alcoholism; substance use disorder; relapses into alcohol and/or substance use disorder, such as group consisting of hyperphagia disorder; obesity; Prader-Willi's syndrome; polycystic ovarian syndrome; increased appetite disorder; obesity in diabetes; pathological food cravings; hypothalamic obesity; Cushing's syndrome and hyperphagia disorder associated with injury to the hypothalamus.

34. The method of treating, alleviating and/or preventing according to claim 30, wherein the steroid-related CNS disorder or disease is obesity.

35. The method of treating, alleviating and/or preventing according to claim 30, wherein the steroid-related CNS disorder is a hyperphagia disorder.

36. The method of treating, alleviating and/or preventing according to claim 30, wherein the steroid-related CNS disorder or disease is Prader-Willi's syndrome.

37. The method of treating, alleviating and/or preventing according to claim 30, wherein the steroid-related CNS disorder or disease is polycystic ovarian syndrome.

38. The method of treating, alleviating and/or preventing according to claim 30, wherein the diabetes is diabetes type II.

39. The method of treating, alleviating and/or preventing according to claim 30, wherein the steroid-related CNS disorder or disease is alcoholism or substance use disorder.

40-41. (canceled)

42. The method of treating, alleviating and/or preventing according to claim 30, wherein the method results in a decrease in bodyweight, optionally wherein e the decrease is seen after 1 to 20 days.

43. The method of treating, alleviating and/or preventing according to claim 30, wherein the compound is administrated intravenously, nasally, per rectum, intravaginally, percutaneously, intramuscularly, or orally.

44. (canceled)

45. The method of treating, alleviating and/or preventing according to claim 30, wherein the compound is administrated in a dose of from about 0.1 to about 300 mg per kg body weight.

46. (canceled)

47. The method of treating, alleviating and/or preventing according to claim 30, wherein said compound provides an antagonistic effect on the effect of γ-aminobutyric acid (GABA) and/or any GABA_{A} receptor modulating steroids (GAMS) on the GABA_{A}-receptor α3 subtype(s).

48-49. (canceled)

50. The method of treating, alleviating and/or preventing according to claim 30, wherein the compound further provides an antagonistic effect on the effect of γ-aminobutyric acid (GABA) and/or any GABA_{A} receptor modulating steroids (GAMS) on the GABA_{A} receptor α1, α2, α4 and/or α5 subtype(s).

51-62. (canceled)
